(19) United States
(12) Patent Application Publication
(10) Pub. No.: US 2022/0213521 A1
(43) Pub. Date: Jul. 7, 2022

(54) SYSTEM AND METHODS FOR GENERATING DYNAMIC MATERIALS HAVING ARTIFICIAL METABOLISM

(71) Applicant: CORNELL UNIVERSITY, Ithaca, NY (US)

(72) Inventors: Dan LUO, Ithaca, NY (US); Shogo HAMADA, Ithaca, NY (US); Kenneth Gene VANCEY, Ithaca, NY (US)

(21) Appl. No.: 17/601,561

(22) PCT Filed: Apr. 3, 2020
(86) PCT No.: PCT/US2020/026680

§ 371 Date: Oct. 5, 2021

Related U.S. Application Data
(60) Provisional application No. 62/829,702, filed on Apr. 5, 2019.

(51) Int. Cl. C12P 19/34; C12Q 1/6844; C12N 9/22; B01L 3/00

(52) U.S. Cl. CPC C12P 19/34; C12Q 1/6844; B01L 2200/16; B01L 3/502715; C12N 9/22

(57) ABSTRACT

This disclosure relates to generation of dynamic materials having an ordered structure and artificial metabolism. The approach disclosed herein allows autonomous and dynamic generation of materials with structural hierarchy by simultaneously coupling both irreversible synthesis (and optionally decomposition) and dissipative assembly processes, but in an artificial fashion. As an exemplary embodiment, DNA-based Assembly and Synthesis of Hierarchical (or “DASH”) materials have been generated. Systems, devices, reagents and methods for generating the materials, as well as additional applications of the present methodology, are disclosed.

### CROSS REFERENCE TO RELATED APPLICATION

This application claims the benefit of priority from U.S. Provisional Application No. 62/829,702, filed Apr. 5, 2019, the entire contents of which are incorporated herein by reference.

### INCORPORATION BY REFERENCE OF SEQUENCE LISTING

The Sequence Listing in an ASCII text file, named as 37255PCT7787 02 PC SequenceListing.txt of 6 KB, created on Mar. 30, 2020, and submitted to the United States Patent and Trademark Office via EFS-Web, is incorporated herein by reference.

### STATEMENT REGARDING FEDERALLY SPONSORED RESEARCH OR DEVELOPMENT

This invention was made with government support under Grant No. EFRI-1331583 and SNM-1530522, awarded by the US National Science Foundation (NSF). The government has certain rights in the invention.

### BACKGROUND

Characteristic properties of life, such as dynamic self-generation of organisms, are sustained by metabolism. Using a flux of matter and energy, molecules are irreversibly synthesized from ingredients and then further dynamically assembled into macromolecules and beyond by series of biological reactions, resulting in the structural hierarchy of life's materials. Although various approaches have been reported to engineer dynamic materials by bioengineering, construction of materials by mimicking metabolism from the ground. up has not been achieved. For instance, engineered living materials allow material generation by life, but the reported approach relies on external living systems such as cells to generate the material. Similarly, other dynamic biomaterials, such as active cytoskeletons, directly use the already-existing metabolism designed by life. In general, although bioengineering approaches have the potential to create novel dynamic biomaterials with sophisticated active behaviors, the current approaches are built upon, and thus fundamentally limited by, life's existing metabolism. Various chemical approaches, especially dissipative self-assembly, have allowed construction of dynamic materials from scratch using chemical reactions.

### SUMMARY OF THE DISCLOSURE

This disclosure is directed to systems and methods for generating dynamic materials having an ordered structure and artificial metabolism. Analogous to the metabolism found in nature, the approach disclosed herein allows autonomous and dynamic generation of materials with structural hierarchy by simultaneously coupling both irreversible synthesis (and optionally decomposition) and dissipative assembly processes, but in an artificial fashion. The artificial metabolism is engineered using molecules and reactions, e.g., biomolecules and hioreactions, but not bound to the restrictions of life itself, due to the bottom-up design of synthesis combined with assembly.

To illustrate the present systems and methodology, and as an exemplary embodiment, DNA-based Assembly and Synthesis of Hierarchical (or “DASH”) materials have been generated, using a mesoscale approach to create dynamic materials from biomolecular building blocks using artificial metabolism (FIG. 1A). Analogous to the materials in living organisms, materials generated by DASH can be synthesized and assembled into pre-coded patterns via anabolism. Furthermore, by integrating anabolism (generation) with catabolism (degeneration), the generated materials can be autonomously degenerated and also regenerated cyclically in situ by combining both generation and degeneration in an ordered fashion, responding to a built-in spatiotemporal feedback. DASH materials having a variety of patterns have been generated. In addition, a DASH material displaying an emergent “locomotion” behavior resembling a slide-mold has been generated. Furthermore, a DASH material having two locomotive bodies displaying an emergent racing behavior has also been generated. The dynamic materials disclosed herein can be utilized as a scaffold for further functionalization to form hybrid materials. In embodiment where the materials are DASH materials, the materials can serve as a platform for providing functions of DNA in a cell-free setting (such as cell-free protein expression). Moreover, the present systems and methods for generating dynamic materials disclosed herein can be applied to pathogen detection.

In one aspect, this disclosure provides a system for generating a material having an ordered structure and artificial metabolism. The system comprises a device and a generation mix, wherein the generation mix is a reagent comprising ingredients for forming a polymer, wherein the device comprises a main chamber designed to permit a directed flow of solution therethrough and to have obstacles which are spaced in a predetermined pattern and are of shapes and sizes to permit generation of vorticity in a directed flow of a solution comprising the generation mix so as to initiate and promote assembly of polymers synthesized in the device thereby forming the material.

In some embodiments, the device is in the form of a flow cell, and the main chamber comprises at least one inlet port, and at least one outlet port. A solution comprising a generation mix can be directed to flow from the at least one inlet port through the main chamber, i.e., through the channels or space between the obstacles, to the at least one outlet port. In some embodiments, the main chamber has a dimension in the micron range, e.g., microfluidic chamber. In some embodiments, the main chamber has a planar shape.

In some embodiments, the system further comprises a degeneration mix, in addition to a generation mix and a device, wherein the degeneration mix comprises reagents that depolymerize the polymer formed by the generation mix.

In some embodiments, the main chamber is designed to permit receiving and directed flow of a solution comprising a generation mix and a solution comprising a degeneration mix. In some embodiments, the main chamber comprises at least two inlet ports for separately infusing a solution comprising a generation mix and a solution comprising a degeneration mix, and at least one outlet port, wherein upon directed flows of the solutions through the main chamber, the process of polymer synthesis and assembly and the process of polymer degeneration occur autonomously and in combination leading to the formation of a material having an ordered structure and artificial metabolism.

In some embodiments, the material generated by the present system has a static pattern, which can take any shape and form. In some embodiments, the material generated has a mobile pattern, e.g., displaying an emergent locomotive behavior, or having two locomotive bodies displaying a racing behavior.

In some embodiments, the device comprises multiple main chambers, which expands the types of patterns for the materials that can be generated.

In some embodiments, the polymer is DNA, and the generated material is also referred to as a DASH material. In some embodiments, a generation mix comprises deoxynucleotides (dNTPs), a template nucleic acid (DNA or RNA), a primer, and a DNA polymerase. In some embodiments, the primer and the template can be annealed prior to being infused into a main chamber. In some embodiments, the template nucleic acid is a circular DNA. In some embodiments, the template nucleic acid is a circular DNA formed from a linear DNA in the presence of a primer and a ligase. In some embodiments, a degeneration mix comprises a deoxynuclease, including, e.g., an exonuclease, an endonuclease, or a combination thereof.

In some embodiments, a generation mix comprises a reagent that produces a detectable signal (e.g., fluorescence) which facilitates the viewing of the generated material.

In a further aspect, this disclosure provides a method for generating a material having an ordered structure and artificial metabolism.

In some embodiments, the method comprises providing a device and a generation mix described herein, supplying a solution comprising the generation mix into the main chamber of the device and directing the flow of the solution through the main chamber, thereby allowing synthesis of polymers and assembly of the synthesized polymers to form the material. In some embodiments, the method utilizes a device that is in the form of a flow cell, and the main chamber comprises at least one inlet port, and at least one outlet port. A solution comprising a generation mix can be directed to flow from the at least one inlet port through the main chamber, i.e., through the channels or space between the obstacles, to the at least one outlet port.

In some embodiments, the method comprises providing a device, a generation mix, and a degeneration mix described herein, supplying a solution comprising the generation mix and a solution comprising the degeneration mix to the main chamber of the device, and directing the flows of the solutions through the main chamber, thereby generating the material. In some embodiments, the main chamber comprises at least two inlet ports for separately infusing a solution comprising a generation mix and a solution comprising a degeneration mix, and at least one outlet port, Wherein upon directed flows of the solutions through the main chamber, the process of polymer synthesis and assembly and the process of polymer degeneration occur autonomously and in combination leading to the formation of a material having an ordered structure and artificial metabolism. The solution comprising the generation mix and the solution comprising the degeneration mix can be infused into the main chamber simultaneously, sequentially, or in a predetermined order.

The material generated can be visualized by naked eye, a camera, a fluorescent microscope, a light microscope, or an electron microscope.

In another aspect, this disclosure provides a system and a method for detecting a nucleic acid of a pathogen. According to this aspect, as device and a generation mix described herein are utilized. However, DNA synthesis and generation of a DASH material occur only when a target pathogen nucleic acid is present in a sample. In some embodiments, a generation mix comprises dNTPs, a template DNA in an initial linear form, a primer, and a DNA polymerase, wherein the template DNA is circularized in the presence of a nucleic acid of a pathogen and a ligase, and the circularized DNA functions as a template for DNA synthesis (e.g., through Rolling Circle Amplification) in the device. In these embodiments, the initial linear form of the template DNA can be brought into contact with a sample being tested and a ligase prior to being supplied to a main chamber, to permit circularization of the template DNA if the target pathogen nucleic acid is present in the sample. Alternatively, the initial linear form of the template DNA, along with other ingredients in a generation mix, a ligase, and a sample are supplied to a main chamber, and the circularization, as well as polymer synthesis and assembly, occur in the main chamber. In other embodiments, a generation mix comprising dNTPs, a primer, and a DNA polymerase, without a template nucleic acid, are utilized. A target nucleic acid of a pathogen, if present in a sample, will serve as the template for polymer synthesis. In still other embodiments, a generation mix comprising dNTPs, a template nucleic acid, and a DNA polymerase, without a primer, are utilized. A target nucleic acid of a pathogen, if present in a sample, will serve as a primer for polymer synthesis. The detection can be accomplished by supplying a solution comprising the generation mix (and the sample in some embodiments) into the main chamber of the device, and directing the flow of the solution through the main chamber, thereby allowing generation and assembly of DNA into a material having an ordered structure and artificial metabolism if the nucleic acid of the pathogen is present in the sample, wherein generation of the material is indicative of the presence of the pathogen nucleic acid. In some embodiments, the nucleic acid of the pathogen is DNA. In some embodiments, the nucleic acid of the pathogen is RNA.

In still a further aspect, a material generated herein is used as a scaffold to generate additional functional materials. In some embodiments, a DASH material is contacted with a DNA-binding reagent supplied into the main chamber of the device in which the DASH material has been formed. In some embodiments, a DNA-binding reagent can be, e.g., avidin, Quantum Dots, and gold nanoparticles. In some enibodiments, a DASH material conjugated. with a DNA-binding reagent can be further functionahzed; e.g., a DASH material conjugated with avidin can be brought into contact with a biotin-conjugated enzyme (e.g., horse radish peroxidase).

In another aspect, a DASH material is used to provide cell-free protein expression.

In still another aspect, this disclosure provides a method for designing obstacles to be placed in a main chamber of a device for generating a material described herein. The method comprises defining a main chamber for generating the material having an ordered structure, defining a pattern of the material to be generated therein; and determining the sizes, shapes and positions of a plurality of obstacles in the main chamber of the device necessary to direct flow of a solution along shortest route within the main chamber and between adjacent obstacles.

### BRIEF DESCRIPTION OF THE DRAWINGS

FIGS. 1A-1K. DASH and the generated materials. (A) Schematics of DASH illustrates anabolic/catabolic pathways of artificial metabolism, (B)-(C) Implementation of DASH. (B) Synthesis of precursor DNA by RCA. (C), Formation of DASH patterns by dissipative assembly using the flow with obstacles in microfluidic devices. (D)-(K) Generated DASH patterns. (D) 1-D lines with maximum 15 μm width, (E), 1-D lines with minimum width. (F) 2-D crosshatch patterns. (G) 2-D drawings (double helix pattern). (H) 2-D drawings (square). (I)-(K) 2-D drawings (D, N, A letter shapes). Dotted lines in subfigures D-F indicate the boundaries of obstacles. See FIGS. 28-42 for further details of the design. Scale bars: (D)-(F) 10 μm, (G) 100 μm, (H) 50 μm, (I)-(K) 100 μm. All flow rates: 0.1 μL/min.

FIGS. 2A-2H. Detailed morphology and hydrodynamics studies of the DASH patterns. A-D Detailed images of the DASH pattern. (A) An overlay of brightfield and green fluorescence channel by confocal fluorescence microscopy, showing both pillars and the DASH patterns. Scale bar=50 μm. (B) Reconstructed 3-D image of A. Dotted line indicates the boundary of the pillar. (C) SEM observation of the DASH pattern. Scale bar=10 μm. (D) Close-up image of (C). Anisotropic networks with embedded spherical structures were observed, Scale bar=1 μm. (E)-(H) Hydrodynamics studies of the DASH pattern generation. (E) a snapshot from the time-lapse video recording of the generation process (experimental result). (F)-(H) CFD simulation results. (F) Flow velocity vector map. Side subfigures represent sections at corresponding locations, indicated by asterisks. (G) Flow velocity heatmap. (H) Flow vorticity heatmap. Dotted arrows indicate the flow direction. All flow rates: 0.1 μL/min.

FIGS. 3A-3L Dynamic behaviors of DASH patterns as machines powered by artificial metabolism. (A)-(C) Sequential generation and degeneration behaviors at a static location. (A) Schematics of the device and the flows. (B) Abstract representation of the behavior by FSA. (C) Snapshots from timelapse video recording (2, 3, 4, 5 hrs) and average fluorescence intensity plot at the location of the DASH patterns. (D)-(F) An emergent locomotion behavior. D, FSA and the program. The design of FSA is expanded by receiving/sending the Flow Altered signal. By using each FSA as a unit, the behavior was programmed by connecting them in a serial fashion via Flow altered signals. A different waiting time until the state transition from Init to Growth (t₁<t₂< . . . <t₆) is used as a parameter. Interpretation represents the equivalent experimental implementation of the program. (E) Details of the final design of the track used in the experiment. (F) Snapshots and center of mass plot (x-axis distance from the origin) representing the locomotion of the body (60 min., 75 min., 92.5 min) in the narrow track. (G)-(I) An emergent racing behavior between two locomotive bodies. (G) A corresponding program for the behavior achieved by placing two locomotion behavior programs (D) in parallel with the signals between two tracks. (H) Interpretation of the program for the actual implementation. (I) Snapshots of the behavior. Both tracks generated the body (75 min.), then started to locomote towards upstream (107.5 min.). Once the symmetry is broken, the body at Track no. 2 leaded the race (dotted line) and started to slow down the body at Track no. 1 by degenerating the body (127.5 min.). After the body at Track no. 2 won the race by reaching the goal (135 min.), the body at Track no. I was completely degenerated (˜180 min.). Flow rates applied: (C) 0.1 μL/min. (F), 0.15 μL/min. for both generation and degeneration mixes.

FIGS. 4A-4E. Other applications of the DASH material. (A) Pathogen DNA/RNA detection by generated DASH patterns. Positive samples at 500 and 50 pM were successfully detected by the generated patterns. Control samples using non-target sequences with 2 bp mismatch resulted with no pattern generation. (B)-(D) Hybrid materials. (B) Fluorescent molecule-conjugated avidin binding. A gradient of two colors was achieved by using 3-inlet device (center flow: Texas Red (red), side flow: FITC (green)). Scale bar=100 μm. (C) Quantum dot attachments mediated by avidin binding. Scale bar=10 μm. (D) DNA-conjugated gold nanoparticle attachments on DASH patterns, observed by darkfield microscopy. Scale bar=10 μm. (E) Cell-free protein expression from the DASH patterns incorporating a reporter gene for sfGFP. Error bars represent standard deviation. All flow rates applied during the DASH pattern generation: 0.1 μL/min.

FIG. 5. Schematics of the generation seed preparation. Template and Primer DNA were mixed with equimolar ratio, then annealed down from 95° C. to 4° C. T4 DNA Ligase was added to hybridized solution and incubated at 4° C. for overnight for the ligation.

FIG. 6. Overall device layout of the DASH device chamber with 500 μm width; 50 μm width channels connected to inlet (square)/outlet (house-shaped) ports.

FIGS. 7A-7H. (A)-(H).s Standard structure elements of DASH patterns. DASH patterns can be simplified into combinations of three elements, such as “Straight”, “Divide”, and “Merge”, described with node-link diagram. Nodes are converted to the pillars or the obstacles; links are converted into actual DASH structures. Actual representation of obstacles can be triangular, square, or other types of shapes that can alter laminar flow and create vorticity at the specific point. Boundaries can be eliminated by considering the symmetry of the flow inside the device.

FIG. 8. Experimental setup of the DASH generation. DASH device was connected to tubing and a syringe. Syringe pump infuses the generation mix at constant flow rate.

FIG. 9. Overall process of DASH data analysis software. The schematic shows the overall flow of the software.

FIGS. 104 10C. CFD simulation of velocity mapping inside 3-chamber device. Flow rate from inlet were set as (A) 0.1155 μL/min.; (B) 0.231 μL/min.; (C) 0.462 μL/min. Note that (C) has a different scale bar due to high flow rate.

FIGS. 11A-11C, CFD simulation of vorticity mapping inside 3-chamber device. Flow rate from inlet were set as (A) 0.1155 μL/min.; (B) 0.231 μL/min.; (C) 0.462 μL/min.

FIG. 12. Sample SNR data used for the generation starting time analysis. Legends correspond with the characteristic flow velocity of the device (Purple (high)—Light blue (low); see Supplementary Text for details). Initial high signals/decrease of the signals were due to the relative low noise in the sample (i.e., no DASH patterns but also low noise value) thus neglected for the measurements. Time points (frame) after the signal ratio started to increase and the first frame surpassed the SNR value of 2.0 (shown with red dotted line) were used as the pattern generation starting time.

FIGS. 134-13B. Flow velocity heatmap of two devices with different pillar-shapes. (A) square-shaped pillar device (#3-1); (B) rhombic-shaped pillar device (#3-2). The overall flow velocity distribution of both heat maps were equivalent.

FIGS. 14A-14B. Flow vorticity heat map of two devices with different pillar-shapes. (A) square-shaped pillar device (#3-1); (B) rhombic-shaped pillar device (#3-2). High vorticity at the side of pillars were only observed with the square-shaped pillar device.

FIG. 15. Pillar-shape comparison of the DASH pattern generation. Red: square-shaped pillar device (#3-1); Blue: rhombic-shaped pillar device (#3-2). The time difference of increasing S/N represents square-shaped pillar devices (higher vorticity) started to generate patterns faster than the rhombic-pillar devices (lower vorticity).

FIGS. 16A-16D. CFD simulation (particle trace from 2 inlets) during generation/degeneration process. (A) before the controlled accumulation occurs, laminar flow creates two regions (red/black); (B) controlled accumulation starts to alter the flow; (C-B) large accumulation at the center mix two types of solutions.

FIG. 17. Repeated generation/degeneration of the DASH patterns at the static location. Two cycles of generation and degeneration were observed (the first peak at approx. 370-400 min., then second peak at approx. 680 min.).

FIG. 18. DNA/RNA detection powered by DASH. The detection process has three steps: recognition, amplification, and readout. DASH pattern generation and recognition achieve both amplification (enzymatic synthesis and flow-based assembly) and readout (mesoscale pattern) following the recognition step using hybridization and ligation.

FIGS. 19A-19B. Signal-to-noise (SNR) ratio of the generated DASH patterns from positive CMV target samples (A). The SNR values were obtained as a power-to-power ratio of the DASH patterns generated in the image (=power from the spatial frequencies corresponding to the DASH patterns/total power from other spatial frequencies). All samples from target concentration of 500 pM and 50 pM succeeded to generate DASH patterns, which corresponded well with this quantitative SNR representation. In the observation, 5 pM samples failed to generate DASH patterns. The corresponding images at the time point of highest SNR from each sample were also shown (B).

FIGS. 20A-20B. Signal-to-noise (SNR) ratio of the generated DASH patterns from non-target samples (A). All samples were below threshold, which corresponded well with our observation (did not generate DASH patterns). The corresponding images at the timepoint of highest SNR from each sample were also shown (B).

FIG. 21. Average intensity vs. Signal-to-noise ratio (S/N) of the DASH patterns with positive and negative target samples. Positive samples at 500 and 50 pM were successfully detected using DASH (arbitrary threshold of S/N=15, shown as gray dotted line), despite similar average fluorescence intensity (Green=500 pM. Cyan=50 pM, Blue=5 pM; Filled circles: pattern detected, Opened circles: pattern undetected). Negative control samples using non-target sequences with 2 bp mismatch (Black=500 pM, Gray=50 pM, Light gray=5 pM) resulted in similar average fluorescence intensities, but with no pattern generation.

FIGS. 22A-22B. DASH-Avidin binding results. (A) Green fluorescence channel showing SYBR Green I (DNA); (B) Red fluorescence channel showing Avidin-Texas Red conjugate. Images show that Avidin is successfully bound to DASH patterns.

FIGS. 23A-23B. DASH-Streptavidin binding results. (A) Green fluorescence channel showing SYBR Green I (DNA); (B) Red fluorescence channel showing Streptavidin-Texas Red conjugate. Images show that Streptavidin was not bound to DASH patterns.

FIGS. 24A-24D. DASH-Quantum dot (Qdots) attachment result. All images were taken using the same capture condition including filters (Ex. 420 nm, Em. 605 nm) and normalization of the images. (A). positive sample after Qdot attachment, before additional 1 hr washing; (B) after 1 hr washing of subfigure a to confirm the binding. Note that overall background was reduced, but the Qdot attachments to the DASH patterns remained; (C) negative control sample without avidin binding. Some Qdots became aggregated but not attaching to the DASH pattern (see high background due to unbound Qdots compared to subfigure a); (D) close-up image of (A). Successful uniform attachment of Qdots to DASH structures was achieved by this method.

FIG. 25. DASH-AuNP patterns. Orange/red tint of DASH patterns represent successful attachment of AuNP to the structure.

FIGS. 26A-26B. Direct observation of CFPE from DASH patterns. a Observation of DASH devices before CFPE. DASH patterns were shown in blue due to their staining with a Hoechst 33342 dye. (A) (left) a device without DASH generation. (right) a device with DASH generation; (B) After CFPE. (left) a device without DASH generation and after CFPE. (right) a device after CFPE with DASH generation.

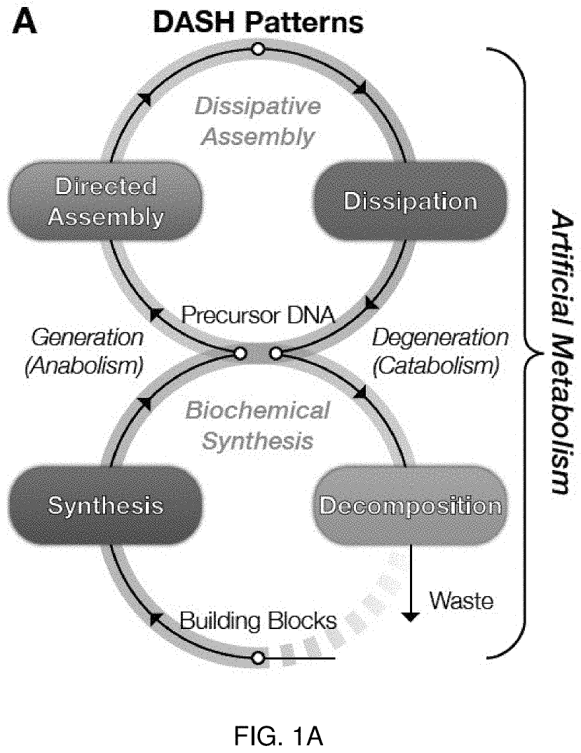

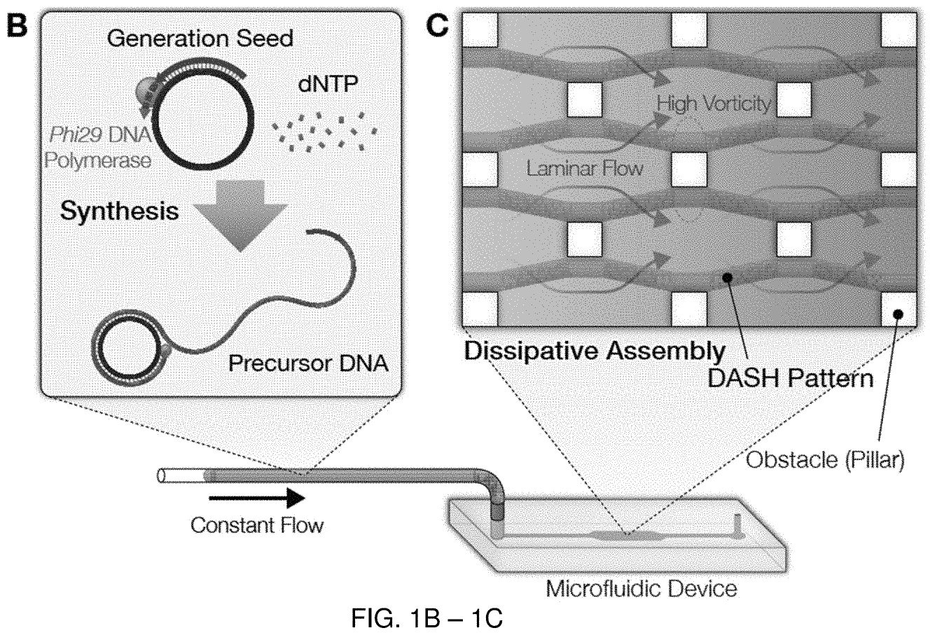

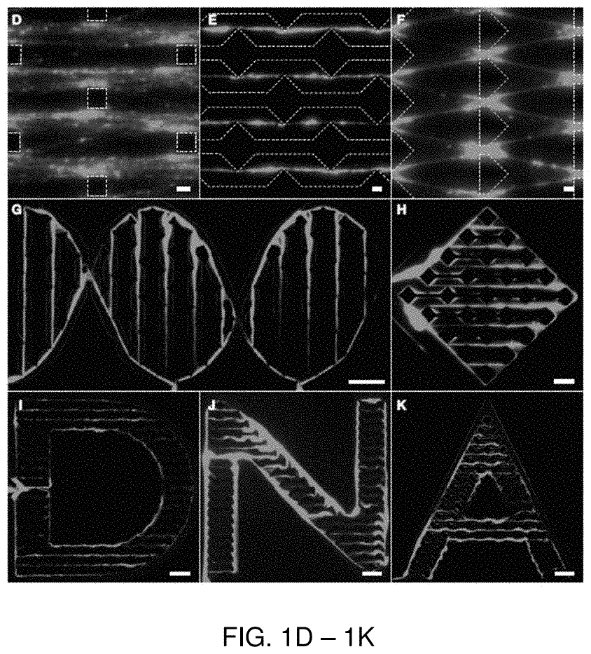

FIGS. 27A-27H. (A)-(H) DASH device and track design catalog. Total 15 designs used. (A)-(H) show brightfield channel images of FIGS. 1D-1K.

FIG. 28A-28C. (A) (C) Design 3-1. Features: 1-1D line (Max 15 μm width). 1 inlet/1 outlet. Pillar-based (imaginary boundaries), 50 μm distance (along with the flow direction) with staggered geometry. 15 μm lateral distance between staggered pillars. In situ observation compatible; shorter total length than typical chamber.

FIGS. 29A-29C. (A) (C) Design 4-5. Features: 1-D line (minimum width). 1 inlet/1 outlet. Obstruction-based (physical boundaries). 50 μm distance between top of triangular obstacles (along with the flow direction), 0 μm lateral distance between top of triangular obstacles. Bypass channel at sides; additional pillars at the entrance and exit of chambers to reduce clogging by overgrow. in situ observation compatible.

FIGS. 304-30C. (A) (C) Design #9-1. Features: Zig-zag lines (creates 2-D crosshatch pattern). 1 inlet/1 outlet. Pillar-based (imaginary boundaries), In situ observation compatible

FIGS. 31A-31C. (A)-(C) Design #18-3, Features: 2-D shapes with “DNA double helix” pattern. linlet/loutlet, each divided into three channels. Obstruction-based (physical boundaries). Typically 50 μm distance between top of triangular obstacles (along with the flow direction). Typically 0 μm lateral distance between top of triangular obstacles. In situ observation compatible.

FIGS. 32A-32C. (A) (C) Design 3-3. Features: 2-D Square shape (1-D lines with square boundaries). 1 inlet/1 outlet. Obstruction-based (physical boundaries). 50 μm distance between top of triangular obstacles (along with the flow direction). 0 μm lateral distance between top of triangular obstacles. 3 Square devices located in tandem (chamber width at the square border varied).

FIGS. 33A-33C. (A)-(C) Design #8-2. Features: 2-D drawings with “Letter D” pattern. 1 inlet/1 outlet. Obstruction-based (physical boundaries), Typically 50 μm distance between top of triangular obstacles (along with the flow direction). Typically 0 μm lateral distance between top of triangular obstacles. In situ observation compatible.

FIGS. 34A-34C. (A)-(C) Design #11-2. Features: 2-D drawings with “Letter N” pattern, 1 inlet/1 outlet. Obstruction-based (physical boundaries). Typically 50 μm distance between top of triangular obstacles (along with the flow direction). Typically 0 μm lateral distance between top of triangular obstacles. In situ observation compatible

FIGS. 35A-35C. (A)-(C) Design #5-1. Features: 2-D drawings with “Letter A” pattern. 1 inlet/1 outlet. Obstruction-based (physical boundaries), Typically 50 μm distance between top of triangular obstacles (along with the flow direction). Typically 0 μm lateral distance between top of triangular obstacles. In situ observation compatible.

FIGS. 36A-34B. (A)-(B) Design #14-2. Features: Zig-zag lines (creates 2-D crosshatch pattern); variant of 9-1 design (identical pillar design). 3 inlet/1 outlet. Inlet 2 divided into two sides. Pillar-based (imaginary boundaries). In situ observation compatible. Optimized design for generation-degeneration experiments.

FIGS. 37A-37B. (A)-(B) Design #20-2. Features: Same device as #14-2, integrated with additional T-junction module. Optimized design for regeneration experiments.

FIGS. 38A-38B. (A)-(B) Design #12-1. Features: 1-D line. 1 inlet/1 outlet, divided into three individual chambers from same source. Variant of 3-1 design (identical pillar design). In situ observation compatible. Optimized design for flow velocity—DASH generation measurement tests.

FIGS. 394-39C. (A)-(C) Design 3-2. Features: A variation of 3-1 (rhombic-shaped pillars, instead of square) for vorticity comparisons [same width of pillars as 3-1, same location, same numbers in order to have equivalent flow velocityl]. 1-D line (rhombic-shaped pillars). 1 inlet/1 outlet. Pillar-based (imaginary boundaries). 50 μm distance (along with the flow direction) with staggered geometry. 15 μm lateral distance between staggered pillars. In situ observation compatible; shorter total length than typical chamber.

FIGS. 40A-40B. (A)-(B) Design #22-3. Features: A variation of #14-2 with varied lateral pillar distances for the locomotion powered by DASH. 1-D line (wide-track). 3 inlet/1 outlet, Inlet 2 divided into two sides. Pillar-based (imaginary boundaries). Track width: 500 μm; 10, 15, 20, 25, 30, 35 μm lateral distance between adjacent pillars. In situ observation compatible.

FIGS. 41A-41B. (A)-(B) Design #23-3. Features: A variation of #22-3 with narrow (150 μm) track width and with two-layered laminar flow for a simpler design for the locomotion powered by DASH. 1-D line (narrow track). 2 inlet/1 outlet. Pillar-based (imaginary boundaries). Track width: 150 μm; 10, 15, 20, 25, 30, 35 urn lateral distance between adjacent pillars. In situ observation compatible.

FIGS. 42A-42B. (A)-(B) Design #23-4. Features: A variation of #22-3 with curved geometry for the locomotion powered by DASH. 1-D line (wide track, U-shaped curve). 3 inlet/1 outlet, Inlet 2 divided into two sides. Pillar-based (imaginary boundaries). Track width: 500 μm; 10, 15, 20, 25, 30, 35 urn lateral distance between adjacent pillars. In situ observation compatible.

### DETAILED DESCRIPTION

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### Materials

This disclosure is directed to generation of materials having an ordered structure and artificial metabolism.

The term “artificial metabolism” is used herein to describe both the features of the present methodology for generating materials and the properties of the materials generated. Analogous to the metabolism found in nature, the methodology disclosed herein allows autonomous and dynamic generation of materials with structural hierarchy by simultaneously coupling both irreversible synthesis/decomposition and dissipative assembly processes, but in an artificial fashion. By integrating anabolism (generation) with catabolism (degeneration), the methodology disclosed herein allows generation of materials which are autonomously degenerated and also regenerated cyclically in situ by combining both generation and degeneration in an ordered fashion, responding to a built-in spatiotemporal feedback. Thus, the materials generated are said to have “artificial metabolism” because the materials have a “metabolism” in the sense that the molecular structure underlying the materials is being generated (anabolism) in an autonomous and dynamic manner by simultaneously coupling irreversible synthesis and dissipative assembly processes, and in embodiments where degeneration is additionally included, the molecular structure underlying the materials is also being degenerated (catabolism) autonomously; in other words, the molecular structure underlying the materials are autonomously and dynamically being generated, degenerated and also regenerated cyclically in situ. Because the processes involved are artificially created, the metabolism described above is said to be an artificial metabolism.

The term “ordered structure” is used herein to describe the hierarchical structural organization of the materials generated herein. For example, a DASH material can have a fibrous structure that is composed of bundles of one-dimensional micron-scaled networks of DNA molecules (as a result of dissipative assembly), which in turn are polymers formed from nanoscale monomers (as a result of polymer synthesis).

The material generated herein can be in any pattern. In referring to a material generated herein, the term “pattern” includes both shape and dimension characteristics, as well as behavioral characteristics. For example, materials of a wide variety of mesoscale patterns and shapes have been generated, from periodically patterned 1-D lines to 2-D arbitrary shapes as illustrated in FIG. 1D-K and FIG. S42. Materials having a mobile pattern, e.g., a DASH material displaying an emergent locomotive behavior, a DASH material having two locomotive bodies displaying a racing behavior, have been generated. As disclosed herein, the pattern of a material can be designed and accomplished based on positioning of obstacles having predetermined sizes and shapes preferably with the aid of Computational Fluid Dynamics (“CFD”) simulations.

The materials having an ordered structure and artificial metabolism can be generated from a variety types of building blocks, i.e., monomers, dimmers, trimers, or oligomers—which can be used to synthesize polymers in situ, and wherein synthesized polymers can also be depolymerized in situ. In some embodiments, a polymer is DNA or RNA.

### Generation Mix

The process of polymer synthesis in situ is accomplished by supplying ingredients needed for synthesis of polymers into a device designed for generating the material described herein. The term “generation mix” is used herein to describe a reagent comprising ingredients needed for polymer synthesis.

In some of the embodiments where the polymer is DNA, a generation mix can include a DNA template (double stranded or single stranded, linear or circular), a primer, deoxynucleotides (dNTPs), and a DNA polymerase. In some embodiments, a DNA template and a primer can be annealed together before use. In some embodiments, the template is a circular DNA which has been circularized in the presence of a primer and a ligase. DNA polymerases suitable for use herein include, but are not limited to, DNA polymerases from prokaryotes (such as DNA Pol I, II, and III from prokaryotes such as E. coli) or from eukaryotes (e.g., DNA Pol α, β, γ, δ, and ϵ), many of which are commercially available. In sonic embodiments, a DNA polymerase is a Phi29 DNA Polymerase which can achieve DNA synthesis by Rolling Circle Amplification (RCA). In some of the embodiments where the polymer is DNA, a generation mix can include a RNA template, a primer, deoxynucleotides (dNTPs), and a reverse transcriptase.

In some of the embodiments where the polymer is RNA, a generation mix can include a DNA template (double stranded or single stranded), a primer, nucleotides (NTPs), a RNA polymerase, and any transcription factors appropriate for inclusion. RNA polymerases suitable for use herein include, but are not limited to, RNA polymerases from prokaryotes or eukaryotes, many of which are commercially available.

In some embodiments, a generation mix can include all the necessary ingredients for synthesis of polymers in situ. In some embodiments, a generation mix can include the necessary ingredients for synthesis of polymers, although synthesis occurs only when a. target nucleic acid is present in a sample to be tested. For instance, in embodiments where the present system and methodology are applied for purposes of detecting a pathogen DNA or RNA, the generation mix can include all the necessary ingredients for DNA synthesis in situ except a template DNA is provided in a linear form and will only be functional as a template after being circularized by a ligase when and only when a target pathogen DNA or RNA is present in the sample. In other embodiments, a generation mix can include ingredients for synthesis of polymers except for a nucleic acid template, such that the pathogen ingredients for synthesis of polymers (DNA or RNA), if present in a sample, will serve as the template to initiate the synthesis of DNA molecules which are then assembled into a material having a predetermined pattern that can be visualized; and if the pathogen DNA or RNA is not in the sample, no synthesis of DNA will occur and no material is formed.

In some embodiments, the generation mix includes a compound that binds to the material generated to permit viewing of the pattern of the material. For example, the generation mix can include a dye that binds to DNA, such as SYBR Green I.

### Degeneration Mix

The process of polymer synthesis depolymerization, or decomposition of synthesized polymers, can be accomplished by supplying ingredients needed for depolymerization of a polymer into a device designed for generating the material described herein. The term “degeneration mix” is used herein to describe a reagent comprising ingredients needed for depolymerization of a polymer.

In some of the embodiments where the polymer is DNA, a degeneration mix can include one or more deoxyribonucleases (DNases), which can be exonuclease or endonuclease (including restriction enzymes), many of which are commercially available. In some embodiments, a degeneration mix can include one or more of Exonuclease Exonuclease III, DNase I and DNase II.

In some of the embodiments where the polymer is RNA, a degeneration mix can include one or more Ribonucleases (RNases), in some embodiments, the RNase comprises an endorihonuclease. In some embodiments, the RNase comprises an exoribonucleases. In some embodiments, the endoribonucleoase is selected from the group consisting of RNase A, RNase ET, RNase III, RNase L,, RNase P, RNase PhyM, RNase T1, RNase T2, RNase U2, and. RNase V. In some embodiments, the exoribonucleoase is selected from the group consisting of polynucleotide phosphorylase (PNPase), RNAse PH, RNAse R, RNAse D, RNAse T, oligoribonuclease, exoribonuclease I, and exoribonuclease H.

### Device

The process of polymer synthesis and assembly, and the process of depolymerization, can be accomplished using a device designed for generating a material having a specific pre-coded pattern.

The device includes a main chamber where a process of polymer synthesis and assembly, and optionally also a process of depolymerization if desired, occur. Generally speaking, the main chamber is not limited to any particular shape or dimension, as long as the chamber permits the supply of a solution comprising a generation mix, and the supply of a solution comprising a degeneration mix where desired, and permits the supplied solution(s) to have a directed flow through the chamber (for example, flow from one end of the chamber having an inlet port to another end having an outlet port) to generate a material of a predesigned pattern, In some embodiments, the device is a microfluidic device, where the main chamber has a dimension in the micron range and takes a substantially planar shape, as shown, for example, in FIGS. 1B-1C.

For generation of a material having an ordered structure and a specific pattern, a main chamber comprises a plurality of obstacles spaced (i.e., positioned) in a predetermined manner based on the pattern of the intended material product, the obstacles being shaped and sized to cause vorticity in a flow of a solution directed through the main chamber. In some examples, the plurality of obstacles are uniform. In some examples, the plurality of obstacles comprises at least one first obstacle and at least one second obstacle, the at least one first obstacle having a different size and/or a different shape than the at least one second obstacle. In some examples, the spacing of the plurality of obstacles is uniform. In some examples, a spacing between at least some of the plurality of obstacles is different than a spacing between others of the plurality of obstacles. In some embodiments, the main chamber does not take a substantially planar shape.

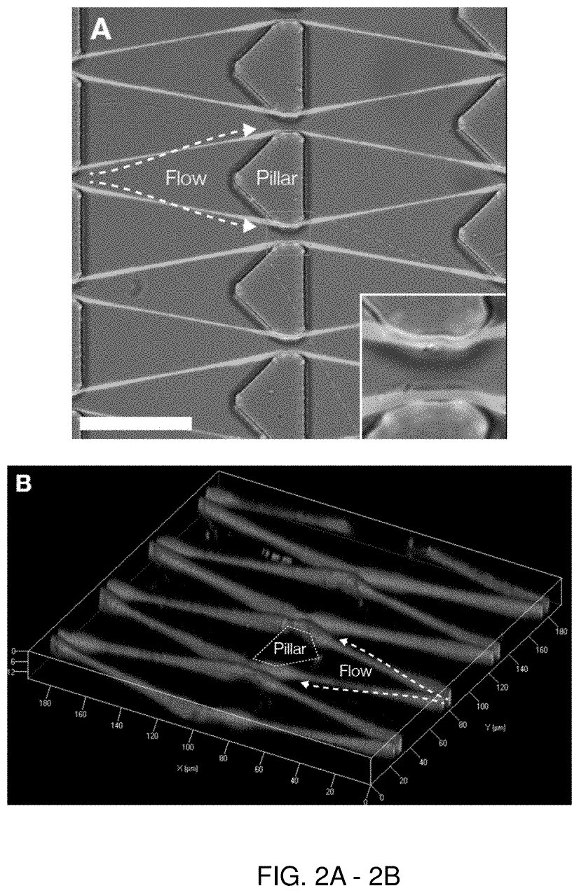

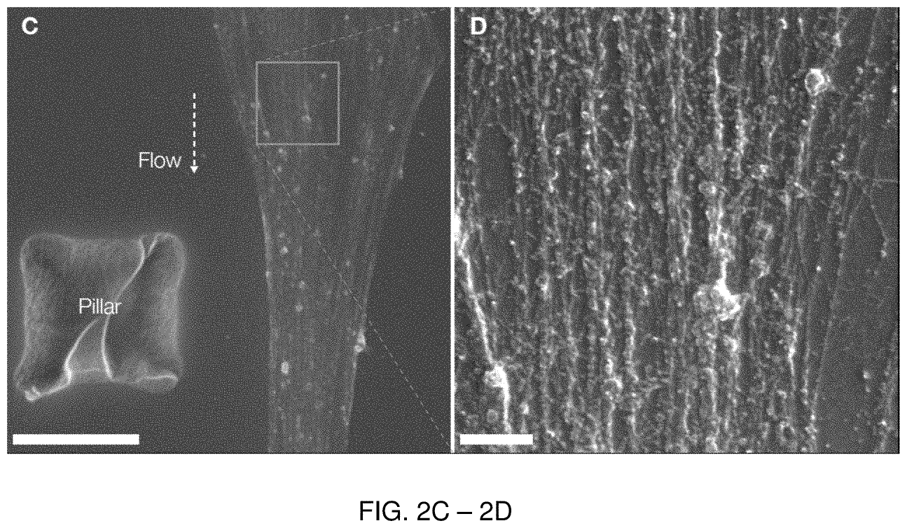

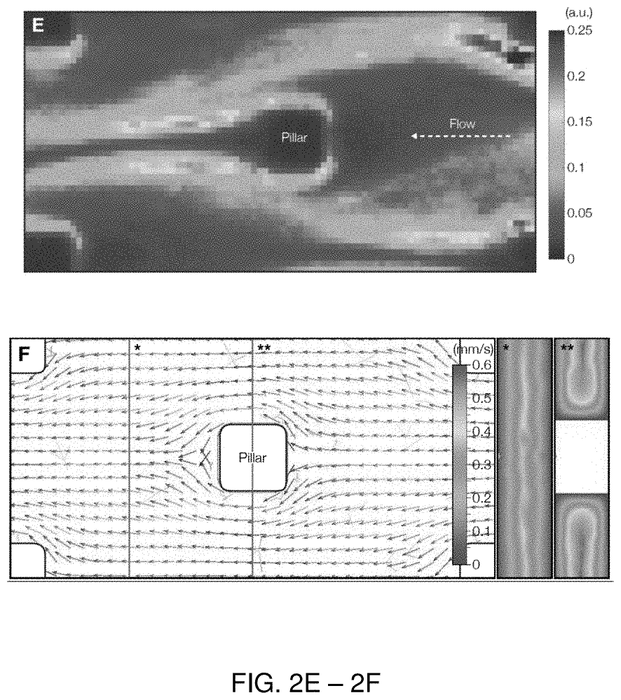

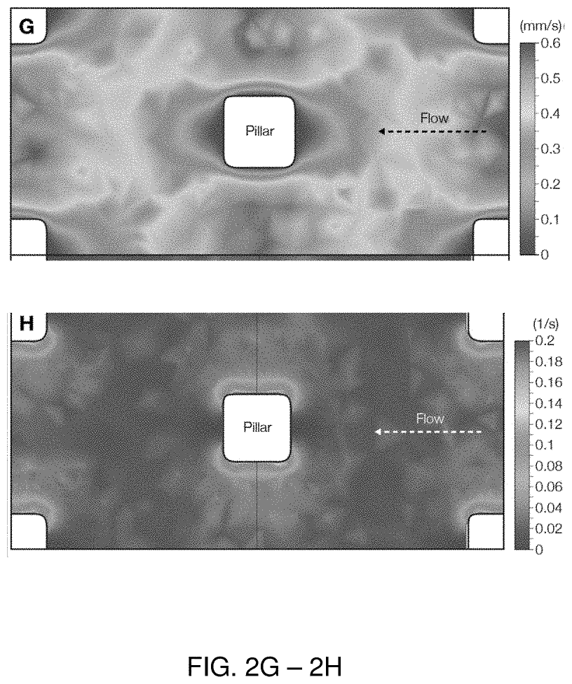

The design of the obstacles, including their shapes, sizes and positions, for generating a material having an ordered structure with a specific pattern, can be accomplished by following the guidelines developed by the inventors based on the observations made from the experiments described herein. In particular, the inventors have found that the mechanism behind the assembly of a final product is a combination of the vortex-induced dynamic formation of new networks of polymers synthesized in situ and the flow-directed redistribution of pre-formed networks. More specifically, the inventors have observed that DNA network formation is initiated from the side edges of the pillars in the middle (i.e. center of the z-axis) of the chamber, and then with additional generations, these DNA networks start to be connected into one continuous fibrous structure between pillars. Further, the inventors have found that the sides of pillars correspond with areas of high vorticity (e.g., as illustrated in FIG. 2H), and that devices having pillar shapes that generate higher vorticity generate DASH patterns faster e.g., as illustrated in FIGS. 13-15). These observations indicate that the flow, particularly the vorticity, is critical in the formation process by locally and dynamically triggering a physical entanglement of DNA into networks at the side of pillars. A higher magnitude of vorticity at the side of pillars leads to an earlier generation starting time. The networks, which have formed, are then redistributed along the direction of flows in the region of highest velocity, to form continuous, fibrous anisotropic structures along the direction of flow (FIGS. 2F, 2G). The thickness of the structures subsequently increase with the gaps between pillars eventually filled with the DNA networks.

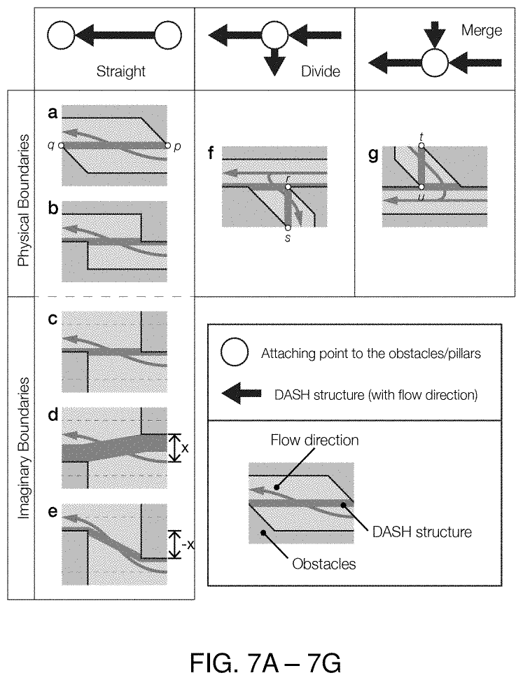

Based on recognition that the assembly of a final product is a combination of the vortex-induced dynamic formation of new networks of polymers synthesized in situ and the flow-directed redistribution of pre-formed networks, and aided by Computational Fluid Dynamics (CFD) simulations, the inventors have established that given a desired pattern of a material, the patterns (i.e., shapes, sizes and positions) of the obstacles in the chamber can be designed based on two simple guidelines: the patterns can be predicted by taking the shortest route within the chamber, and by connecting adjacent pillars (obstacles), both in accordance to the direction of flow. As illustration, microfluidic devices have been designed by a simple combinatory rule using seven types of structural units (FIG. 7). A combination of units codes the location of pillars along with the routes of flow in the device to satisfy both guidelines, enabling a general design strategy for the DASH patterns. FIG. 27 illustrates a number of patterns of materials, and the underlying patterns (shapes, size and positions) of obstacles for generating materials having such patterns of materials. Generally speaking, the obstacles may be considered as a plurality of nodes connected by links, with a “node” being a region with high vorticity which geometry-wise, is an area corresponding to a side “tip or edge” of the obstacles (e.g., points p,q,r,s mentioned in the FIGS. 7A-7G, side edges of square-shaped pillars, etc.), and “links” are the shortest connections between those points along with the direction of flow.

In some embodiments, the pattern of a material, once visualized, has an appearance that is static, i.e., not mobile. For example, DASH materials have been generated in a wide variety of patterns, for example, from periodically patterned 1-D lines to 2-D arbitrary shapes (FIGS. 1D-K and FIG. 27).

In some embodiments, the pattern of a material is mobile (i.e., moving).

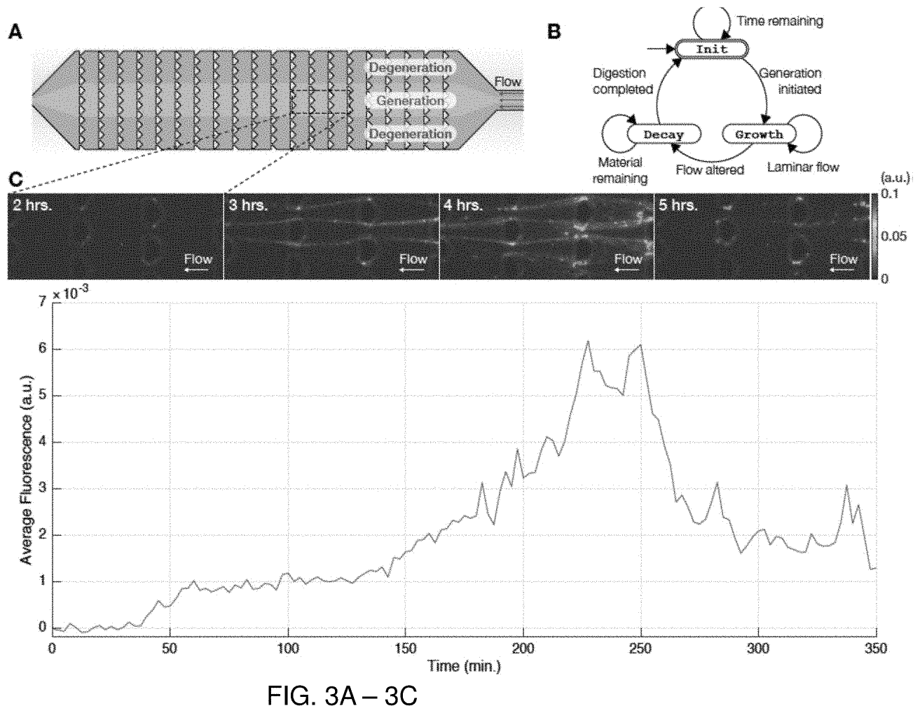

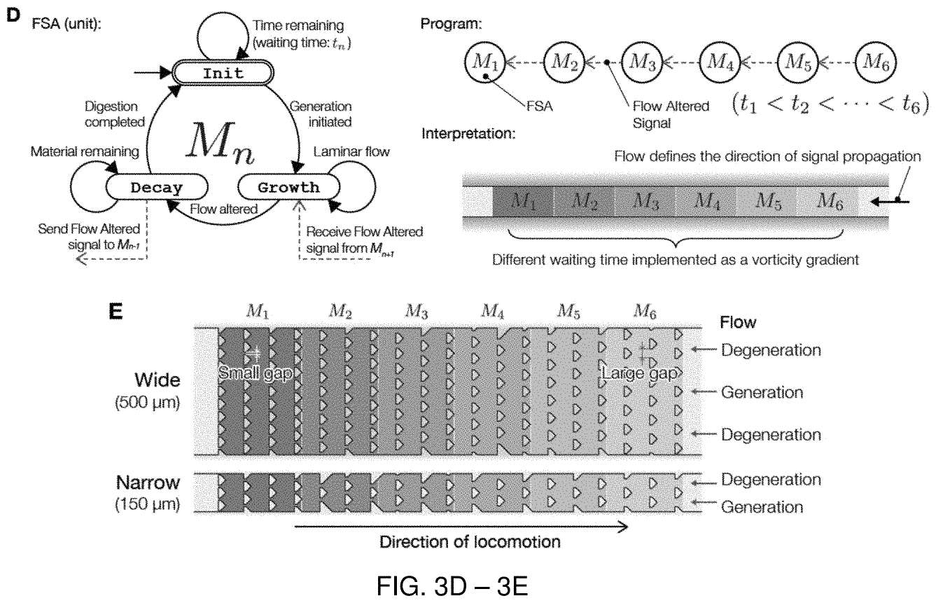

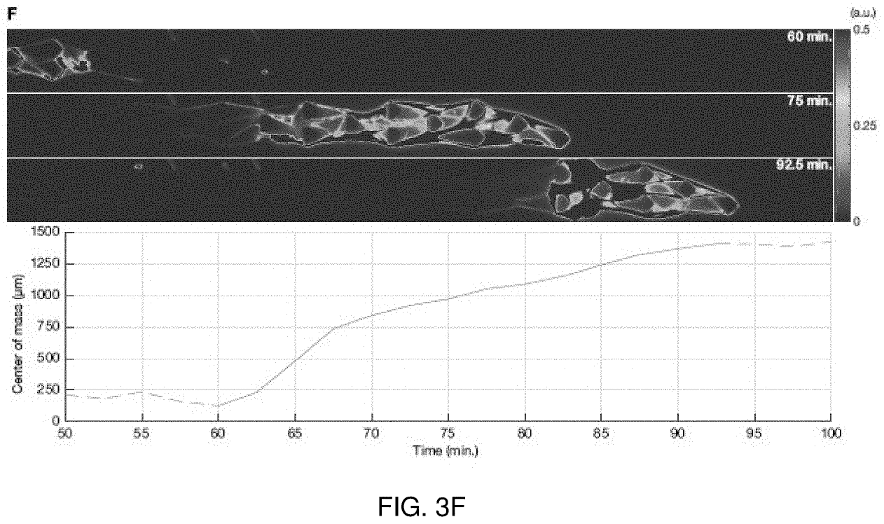

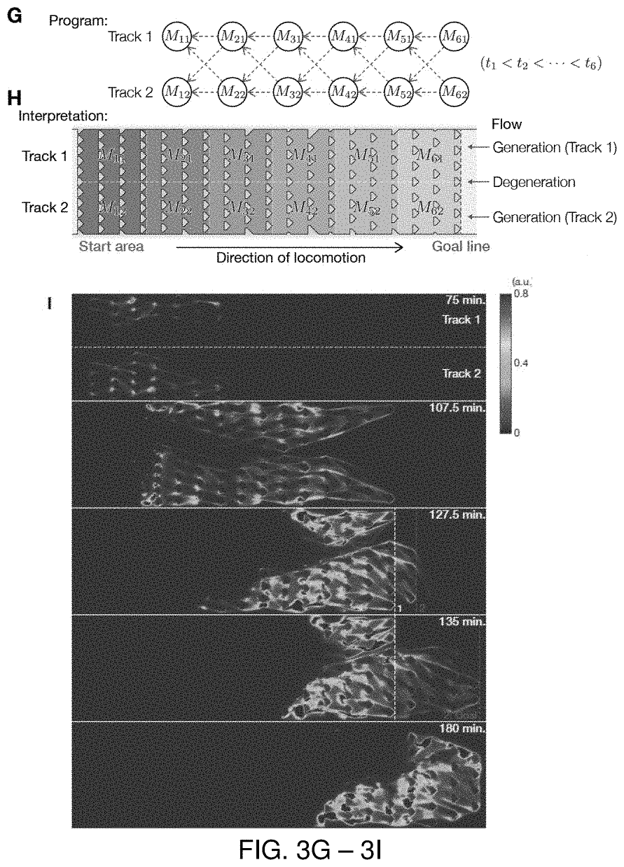

In some embodiments, a material that displays an emergent locomotive behavior. As illustration, the inventors have demonstrated generation of a DASH material that displays an emergent locomotive behavior. The overall behavior of the material is described by using a finite-state automaton (“an FSA”) with three states, Init, Growth, and Decay, with autonomous and sequential state transitions (FIG. 3B). This abstraction with discrete states and state transitions, similar to the methods used in mechanical robots, has allowed interpretation of the overall behavior of the material as a machine, and thus enabled further programming of the behavior. Notably, the state transition between Growth and Decay was switched by a spatiotemporal feedback. A microfluidic device having three inlet ports and one outlet port was used to illustrate this embodiment (FIG. 16). A generation mix was infused into the device through the inlet port in the middle, whereas a degeneration mix was infused into the device through the other two inlet ports on the outside. Initially, all three solutions flowing into the device remained laminar

(“Init” state). Thus, the degeneration mix was kept separated from the generation mix, and the anabolic process started at the center of the device (“Growth” state). Gradually, accumulation by redistributed DNA networks started to fill the gap between the pillars, substantially altering the flow dynamics. This spatiotemporal feedback allowed both the generation and degeneration solutions to be mixed, thus triggering the state transition. The catabolic process now started to dominate, and finally the materials were degenerated (“Decay” state). Experimentally, the inlet channels containing generation and degeneration mixes were prepared as predefined tracks (FIG. 3E), with the gap between adjacent pillars being tuned from small (downstream) to large (upstream) in a region-by-region fashion along the track. Each region corresponds to each FSA (Init, Growth, and Decay). The size of the gap which defines the magnitude of vorticity represents a parameter (waiting time) in each unit to trigger the state transition from Init to Growth. As programmed, this vorticity gradient elicited a spatiotemporal delay of the transition from Init to Growth state, starting from the downstream region of the track. In short, the direction of the locomotion is experimentally interpreted as a gradient in the magnitude of vorticity under the constant flow rate. The direction of locomotion was deliberately programmed to be against the flow direction in all examples. After the autonomous generation started at the downstream region and the body constituted by the DASH patterns started to grow, a spatial feedback due to the generated patterns triggered the transition to Decay state, also starting from downstream. The transition to the Decay state is also propagated to the downstream regions due to the flow, assuring that catabolism would dominate in these regions. At the same time, the transition from Init to Growth state continued towards the upstream region (i.e. down the vorticity gradient). As a result, an overall locomotion behavior of the body along the track against the direction of flow emerged as programmed by a series of FSA.

In some embodiments, a material displays an emergent an emergent racing behavior of two competing bodies. To illustrate these embodiments, the inventors have demonstrated generation of a DASH material having two locomotive bodies that display an emergent racing behavior by programming two series of FSA (FIG. 3G). Each series was designed in the same manner as in the example of emergent locomotion behavior; in addition, a simple interference was added between two locomotive bodies. Specifically, the state transition signal from Growth to Decay can also interfere between tracks; and the faster moving body can affect and alter the state of another track to Decay, thus “slowing down” the locomotion of the body at the other track by triggering the degeneration. Experimentally, the design was implemented by simply inverting the types of flow (the generation mix on the outside channels, and the degeneration mix on the inside channel). There is no boundary between two tracks, and thus the altered flow at one track can also affect the state of the other track. The result illustrates a competing race between two bodies with the winner at Track no. 2 (FIG. 3I). As programmed, once the symmetry between two bodies was broken presumably due to the randomness in flow and the body, Decay state caused by the leading body at Track no. 2 affected the body at Track no. 1, resulting in the degeneration of the body. After the body at Track no. 2 reached the goal, ultimately the behavior ended with a complete degeneration of the body at Track no. 1.

### Methods and Device Set Up

To generate a material, a solution comprising a generation mix is supplied to the main chamber of a device and is directed to flow through the main chamber of the device, along the channels (i.e., the space) between obstacles. In some embodiments, a solution comprising a generation mix is infused through an inlet port into the main chamber towards an outlet port. The velocity of the flow can be controlled by various means, e.g., through a pump connected to the inlet or outlet port(s). Once a solution is supplied to the main chamber, the simultaneous processes of synthesis and assembly of polymers occur and continue autonomously.

In some embodiments, a solution comprising a generation mix and a solution comprising a degeneration mix are both supplied to the main chamber of a device and are directed to flow through the main chamber. The two solutions can be supplied to the main chamber simultaneously, sequentially, or in a predetermined order, to permit generation of various patterns. In some embodiments, the two solutions are infused through separate inlet ports, arranged in various manners, e.g., through three inlet ports with the middle port used for the generation mix and the outside ports for the degeneration mix, or vice versa, Once the solutions are supplied to the main chamber, the simultaneous processes of synthesis/decomposition and assembly of polymers occur and continue autonomously,

The materials generated can be visualized by various means, including for example, by naked eye, a camera, a fluorescent microscope (where the polymer is conjugated with a fluorescent compound, for example), a light microscope, or an electron microscope.

### Additional Applications of DASH Materials

In a further aspect, the present methodology for generating a DASH material has been applied to pathogen detection. According to this aspect of the disclosure, a generation mix can prepared to provide selective amplification and generation of a DASH material when and only When a target pathogen DNA or RNA sequence is present in a sample.

In some embodiments, a generation mix comprises dNTPs, a template DNA in an initial linear form, a primer, and a DNA polymerase, wherein the template DNA is circularized in the presence of a nucleic acid of a pathogen and a ligase, and the circularized DNA serves as a template for DNA synthesis (e.g., through Rolling Circle Amplification) in the device. In these embodiments, the initial linear form of the template DNA can be brought into contact with a sample being tested and a ligase, prior to being supplied to a main chamber, to permit circularization of the template DNA if the target pathogen nucleic acid is present in the sample. Alternatively, the initial linear form of the template DNA, along with other ingredients in a generation mix, a ligase, and a sample are supplied to a main chamber, and the circularization, as well as polymer synthesis and assembly, occur in the main chamber. In some embodiments, a generation mix may contain a template DNA, dNTPs and a DNA polymerase, but without a primer needed to initiate DNA synthesis; and a target pathogen DNA, if present in a sample, will serve as a primer needed to initiate DNA synthesis when combined with the generation mix. In some embodiments, a generation mix may contain a primer, dNTPs and a RNA-dependent DNA polymerase (or reverse transcriptase), but without a template needed to initiate DNA synthesis; and a target pathogen RNA. If present in a sample, will serve as a template needed to initiate DNA synthesis when combined with the generation mix.

A solution comprising a generation mix and a sample is infused into the main chamber of a device described hereinabove, and a DASH material is formed when and only when a target pathogen DNA or RNA is present in the sample. A control test, where the device is supplied with a solution containing a generation mix and the target pathogen DNA or RNA can be perform in parallel.

This aspect of the disclosure can be used to detect a DNA or RNA of any pathogen, including, for example, a bacterium, a fungus, or a virus. A sample suitable for use includes any sample containing a suspected pathogen, including an environmental sample (e.g., soil, water), an agricultural or food product (e.g., fruits, vegetables, and poultry), a sample obtained from humans or non-human animals (e.g., mouth or nose swab samples, blood samples, urine or fecal sample, etc.). A sample can be a processed sample, e.g., by subjecting an original sample to centrifugation, cell lysis, fractionation, or any other procedural that may facilitate release, purification, and/or concentration of a target pathogen DNA or RNA, prior to being infused into a device.

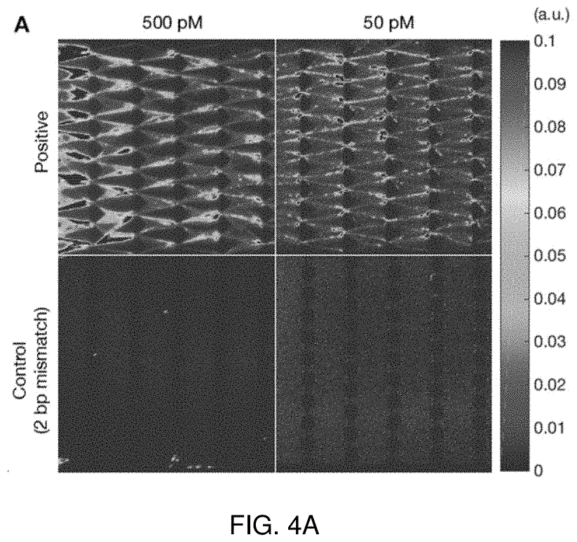

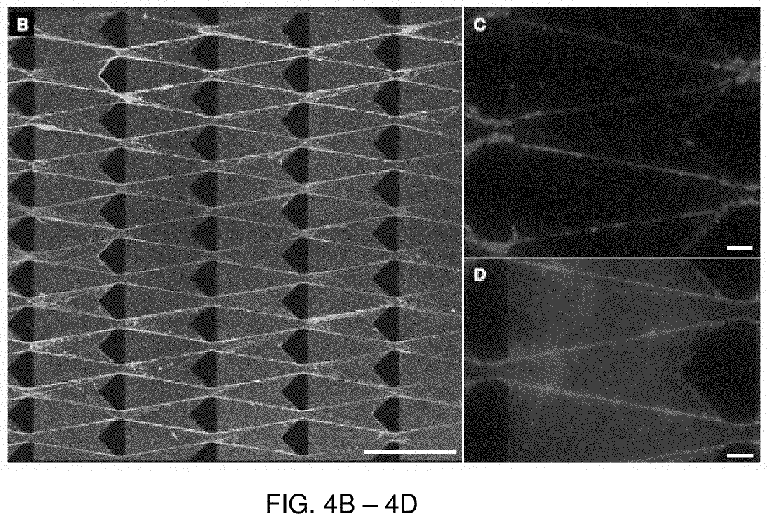

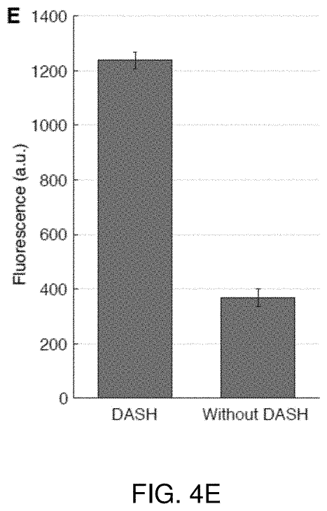

To illustrate this aspect of the disclosure, the inventors have chosen a target sequence taken from Cucumber Mosaic Virus (CMV) as a model pathogen, and have demonstrated successful detection of the target at 500 and 50 pM concentrations by recognizing the self-generated DASH patterns (FIGS. 4A and 19-21). In contrast, a negative control target sequence with a mismatch of only 2 by did not generate the patterns, demonstrating the specificity of the detection method.

In still another aspect, a material generated is used as a scaffold to generate additional functional materials. For example, a DASH material can serve as a versatile mesoscale scaffold for generating a diverse range of functional nanomaterials beyond DNA.

In some embodiments, a DASH material, once formed, is contacted with a reagent that binds to DNA. The reagent can be infused into the main chamber of the device in which the DASH material has been formed, and can be a range of materials including inorganic nanoparticles such as avidin, Quantum Dots, and gold nanoparticles, A DASH material conjugated with any of these reagents can be further functionalized. For example, a DASH material conjugated with avidin, can be contacted with a biotin-conjugated enzyme e.g., horse radish peroxidase).

In some embodiments, the DNA molecules within a DASH material are used to produce proteins encoded by the DNA molecules in a cell-free fashion and in a spatiotemporally-controlled manner. This can be accomplished by supplying a solution comprising a cell-free protein expression system to the device in which the DASH material has been formed. Cell-free protein expression systems are known in the art and also commercially available. In some embodiments, the cell-free protein expression system comprises a lysate that comprises components necessary for protein synthesis. In some embodiments, the components necessary for protein synthesis comprise tRNA, ribosomes, amino acids, initiation, elongation and termination factors. In some embodiments, the cell-free protein expression system comprises an E. coli lysate. In some embodiments, the cell-free protein expression system comprises a wheat germ lysate. In some embodiments, the cell-free protein expression system comprises a rabbit reticulocyte lysate. In some embodiments, the cell-free protein expression system comprises a HeLa-based lysate.

In some embodiments, a cell-free protein expression system also includes a primer which is designed to bind to and activates a promoter in the DNA of the DASH material, thereby initiating transcription of the gene encoding the desired protein, and subsequent protein production.

The specific examples listed below are only illustrative and by no means limiting.

### EXAMPLES

### Example 1

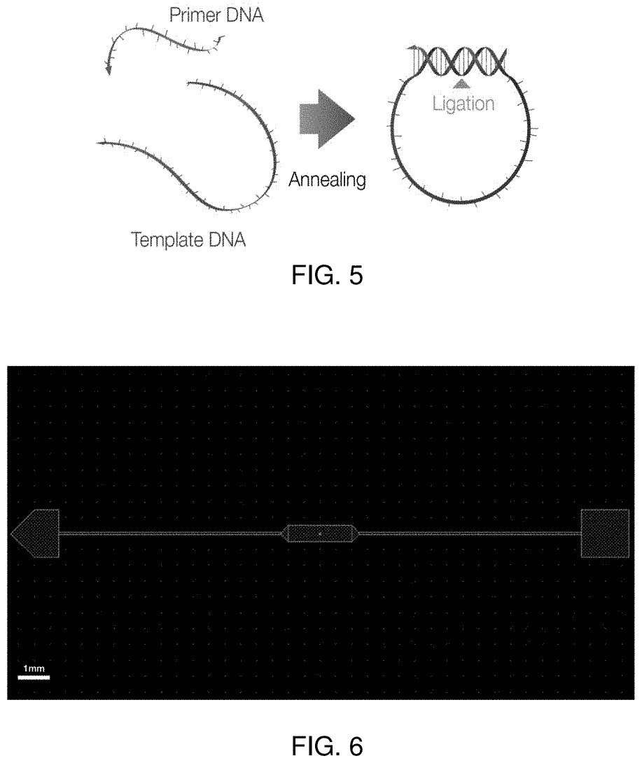

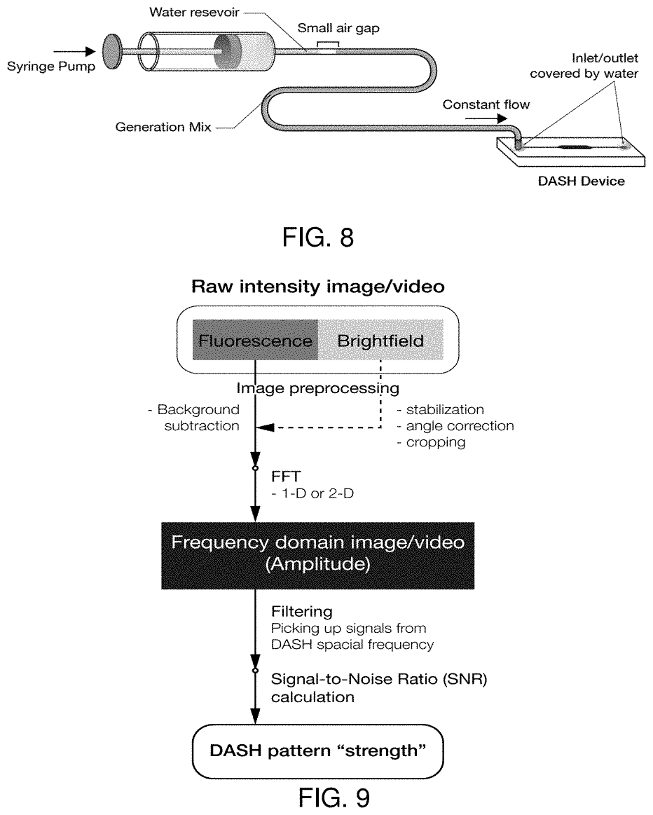

The anabolic pathway of DASH consists of two key simultaneous and autonomous processes to represent the concept of artificial metabolism: 1) biochemical synthesis of DNA molecules as a precursor of the material accomplished by an in situ enzymatic reaction, and 2) dissipative assembly of precursors to form the material with pre-coded patterns and shapes by flow. Specifically, in the process of precursor synthesis, in situ DNA synthesis was accomplished by Rolling Circle Amplification (RCA) using Phi29 DNA Polymerase, in a generation mix which also contained seeds (DNA templates with primers) and building blocks (FIG. 1B and FIG. 5). During the process of dissipative assembly, specific patterns were directly assembled from precursor DNA using flow in microfluidic devices. Specifically, the generation mix was continuously infused into a microfluidic device with precisely spaced obstacles to assemble precursor DNA into pre-coded, specific patterns (FIG. 1C, FIG. 6, and FIG. 8). DASH thus achieved the aforementioned anabolic pathway by autonomously generating the material with structural hierarchy across scales: starting from nanoscale building blocks, to polymer precursors, to micron-scaled networks (hydrogels), and finally to mesoscale patterns and shapes, all via simultaneous processes.

Experimentally, a wide variety of mesoscale patterns and shapes, from periodically patterned 1-D lines to 2-D arbitrary shapes, were generated to demonstrate the anabolic pathway of the material (FIG. 1D-K and FIGS. 27A-27H). The pathway enabled autonomous generation of patterned materials by organizing one-dimensional, micron-thick fibrous DNA networks. Aided by Computational Fluid Dynamics (CFD) simulations, it was found that these DASH patterns could be deterministically designed based on two simple guidelines: the patterns were predicted by taking the shortest route within the channel, and by connecting adjacent pillars (obstacles), both in accordance to the direction of flow. To simplify the process, microfluidic devices were designed by a simple combinatory rule using seven types of structural units (FIGS. 7A-7G). A combination of unit codes the location of pillars along with the routes of flow in the device to satisfy both guidelines, enabling a general design strategy for the DASH patterns.

Confocal fluorescence microscopy and scanning electron microscopy (SEM) revealed detailed morphologies of the generated material. Confocal microscopy of 2-D crosshatch patterns showed that the materials were formed in the middle of the microfluidic chamber (away from top and bottom of the chamber) with a fibrous morphology (FIGS. 2A and 2B). SEM observations revealed more detailed morphologies (FIGS. 2C and 2D): the fibrous structures were made of anisotropic bundles of DNA networks in which the orientation coincided with the direction of flow. Here a device with 1-D line patterns was chosen due to its easier transfer for the observation. Most of the materials were localized at the side edge of the pillars parallel to the flow direction and in the space between them, with little discernible DNA wrapped around the pillars. In addition, SEM observations revealed spherical structures with an average diameter of ˜0.3 μm embedded inside the networks, similar to earlier reports on physically-entangled DNA hydrogels (J. B. Lee et al, Nature Nanotechnology. 7, 816 820 (2012)). However, anisotropic networks were evident here between the spherical structures but not in the DNA hydrogel presumably due to the directional flows.

To better understand the mechanism behind the pattern generation by DASH, time-lapse videos were recorded and quantified (FIG. 2E), The existence of elapsed time between flow initiation and the onset of the pattern generation suggested the network formation depended on a minimum molecular weight of synthesized precursor DNA (e.g., an estimated Molecular Weight of 3.3×10⁷ in the case of 2-D crosshatch patterns with 5 nM. of generation mix). Interestingly, we observed that network formation was initiated from the side edges of the pillars in the middle (i.e. center of the z-axis) of the chamber, and then with additional generations, these DNA networks started to be connected into one continuous fibrous structure between pillars. If the dominant mechanism was wrapping DNA networks around the pillars, the fibrous morphology should have started from the upstream edge instead of the side edges of the pillars, and the overall pattern generation should have started from the upstream region of the device. The observation disclosed herein indicated otherwise; the side edges of the pillars were the main places where the assembly was initiated. Thus, the inventors hypothesized that the assembly mechanism of the DASH patterns was a combination of two processes: the formation of the DNA network triggered at the side edges of the pillars, and the redistribution of preformed. networks (both in situ and in flowing solution) into continuous, fibrous anisotropic structures along the direction of flow. The time-lapse images illustrated that the thickness of the structures increased in the later stages with the gaps between pillars eventually filled with the DNA networks. This additional thickening strongly suggested that the redistribution of excess DNA networks formed in solution happened later in the process of the pattern formation rather than earlier.

In order to investigate the underlying mechanisms of the assembly process, the inventors first performed CFD simulations and then experimentally verified them from two aspects: the formation of new networks and the redistribution of preformed networks into a fibrous morphology (FIGS. 2F-2H). The DASH patterns with 1-D lines were chosen for the comparison due to their geometric simplicity. For the formation, it was found that the sides of the pillars corresponded with areas of high vorticity (FIG. 2H). These results along with the sensitivity measurements (FIGS. 10A-10C, FIGS. 11A-11C, and FIG. 12) and a pillar-shape comparison experiment (FIGS. 13A-13B, FIGS. 14A-14B and FIG. 15) suggested that the flow, particularly the vorticity, was critical in the formation process by locally and dynamically triggering a physical entanglement of DNA into networks at the side of pillars. In short, a higher magnitude of vorticity at the side of pillars led to an earlier generation starting time. Similar vorticity-triggered structure formation observed in biofilms and proteins also supported this hypothesis. For the redistribution, an overlay of time-lapsed videos with simulations of flow velocity clearly showed that the fibrous structure was indeed formed along the direction of flows in the region of highest velocity, consistent with the redistribution mechanism (FIGS. 2F-2G). Therefore, the mechanism behind the assembly was most likely a combination of the vortex-induced dynamic formation of new networks and the flow-directed redistribution of pre-formed networks.

Integrating the above anabolic generation process with a catabolic degeneration process via DNA hydrolyzing enzymes further expanded the metabolic pathways of the artificial metabolism. First, both anabolic and catabolic pathways were used to induce sequential generation and degeneration of the pattern at a static location. Here, the DASH patterns were autonomously generated and then synchronously and autonomously degenerated by a combination of enzymatic reactions and flows (FIGS. 3A-3C), The reagents required for both generation and degeneration were simultaneously flowed into the microfluidic device. Crucially, once the flow started, both the processes of generation and degeneration were executed without any external manipulation. A 3-inlet microfluidic device was used with the center inlet containing the generation mix with DNA polymerase, while the inlets at either side contained the degeneration mix with the DNA hydrolyzing enzyme, DNase I. The overall behavior of the material was described by using an FSA with three states, Init, Growth, and Decay, with autonomous and sequential state transitions (FIG. 3B). This abstraction with discrete states and state transitions, similar to the methods used in mechanical robots, allowed interpretation of the overall behavior of the material as a machine, and thus enabled further programming of the behavior mentioned below. Notably, the state transition between Growth and Decay was switched by a spatiotemporal feedback. CFD simulation illustrated the hydrodynamics during the process (FIGS. 16A-16D). Initially, all three solutions flowing into the device remained laminar (Init). Thus, the degeneration mix was kept separated from the generation mix, and the anabolic process started at the center of the device (Growth). Gradually, accumulation by redistributed DNA networks started to fill the gap between the pillars, substantially altering the flow dynamics. This spatiotemporal feedback allowed both the generation and degeneration solutions to be mixed, thus triggering the state transition. The catabolic process now started to dominate, and finally the materials were degenerated (Decay). Additional experimental tests showed that the sequential occurrence of generation and degeneration (cyclical regeneration) could be autonomously repeated at least two times when the DNA synthesis time was kept constant (FIG. 17), demonstrating that both anabolic and catabolic pathways could he seamlessly integrated and regulated in a regenerative fashion without any interference from outside.

Based on the dynamic generation and degeneration behaviors of the material at the static location, a locomotive behavior powered by artificial metabolism using DASH was programmed (FIGS. 3D-3F). Inspired by the shapes and migrating behaviors of pseudoplasmodia (slug) of the cellular slime mold Dictyostelium discoideum (J. T. Bonner, American Journal of Botany. 31, 175 (1944)), a behavior was programmed in which a slug-like body is first generated by autonomous growth of DASH patterns, followed by autonomous locomotion of the body along a track against a constant flow. The locomotion was realized as an emergent behavior based on continuous polarized regeneration: the front-end generates its body, and the back-end degenerates itself. At the abstract design level, the behavior was programmed by expanding the FSA introduced above in a serially-connected manner (M₁ to M₆), regarding each FSA as an autonomous and modular unit (FIG. 3D). Each unit (Mₙ) can accept a “Flow altered” signal from adjacent unit that triggers the state transition from Growth to Decay, and can also propagate the signal to the next (Mₙ₋₁). The locomotion behavior was programmed by setting different waiting time (t₁<t₂< . . . <t₆) until the state transition between Init and Growth is triggered. Growth of each FSA starts from M₁ according to the waiting time. Once the unit Mₙ changes its state to Decay due to its internal feedback, the Flow altered signal is propagated to the adjacent unit Mₙ₋₁ and beyond, ensuring the state transition at the back-end of the body. As a result, the direction of locomotion is represented as spatiotemporal delay of Growth and sequential state transition to Decay. Experimentally, similar multi-inlet channels containing generation and degeneration mixes were prepared as predefined tracks for the behavior (FIG. 3E). Here the gap between adjacent pillars was tuned from small (downstream) to large (upstream) in a region-by-region fashion along the track. Each region corresponds to each FSA. The size of the gap which defines the magnitude of vorticity represents a parameter (waiting time) in each unit to trigger the state transition from Init to Growth. As programmed, this vorticity gradient elicited a spatiotemporal delay of the transition from Init to Growth state, starting from the downstream region of the track. In short, the direction of the locomotion is experimentally interpreted as a gradient in the magnitude of vorticity under the constant flow rate. We also emphasize here that the direction of locomotion was deliberately programmed to be against the flow direction in all examples, After the autonomous generation started at the downstream region and the body constituted by the DASH patterns started to grow, a spatial feedback due to the generated patterns triggered the transition to Decay state, also starting from downstream. The transition to the Decay state is also propagated to the downstream regions due to the flow (represented as a “Flow altered” signal sent to Mₙ₋₁), assuring that catabolism would dominate in these regions. At the same time, the transition from Init to Growth state continued towards the upstream region (i.e. down the vorticity gradient). As a result, an overall locomotion behavior of the body along the track against the direction of flow emerged as programmed by a series of FSA. The behavior was experimentally observed in both straight (wide and narrow widths) and curved tracks, illustrating the design flexibility of the trajectory. The locomotion velocity was measured as 2.3 mm/hr, with narrow tracks (FIG. 3F).

To further demonstrate the application of the material as a machine, the design was expanded by utilizing the power of abstracted programming method to achieve an emergent racing behavior of two competing bodies by two series of FSA (FIG. 3G). Each series (M₁₁ to M₆₁, M₁₂ to M₆₂) is designed in the same manner as the previous emergent locomotion behavior; in addition, here a simple interference was further added between two locomotive bodies. Specifically, the state transition signal from Growth to Decay can also interfere between tracks (denoted by the arrows between two series of FSA); the faster moving body can affect and alter the state of another track to Decay, thus “slowing down” the locomotion of the body at the other track by triggering the degeneration. This program can be interpreted as two tracks representing two series of FSA located side by side without any physical boundaries between each other (FIG. 3H). Experimentally, the design was implemented by simply inverting the types of flow (the generation mix on the outside, and the degeneration mix on the inside) in a wide-width track introduced in the previous section. Since there is no boundary between two tracks, the altered flow at one track can also affect the state of the other track, The result successfully showed a competing race between two bodies with the winner at Track no. 2 (FIG. 3I). As programmed, once the symmetry between two bodies was broken presumably due to the randomness in flow and the body, Decay state caused by the leading body at Track no. 2 affected the body at Track no. 1, resulting in the degeneration of the body (see the snapshot at 127.5 min.). After the body at Track no. 2 reached the goal (135 min.), ultimately the behavior ended with a complete degeneration of the body at Track no. 1 (180 min.).

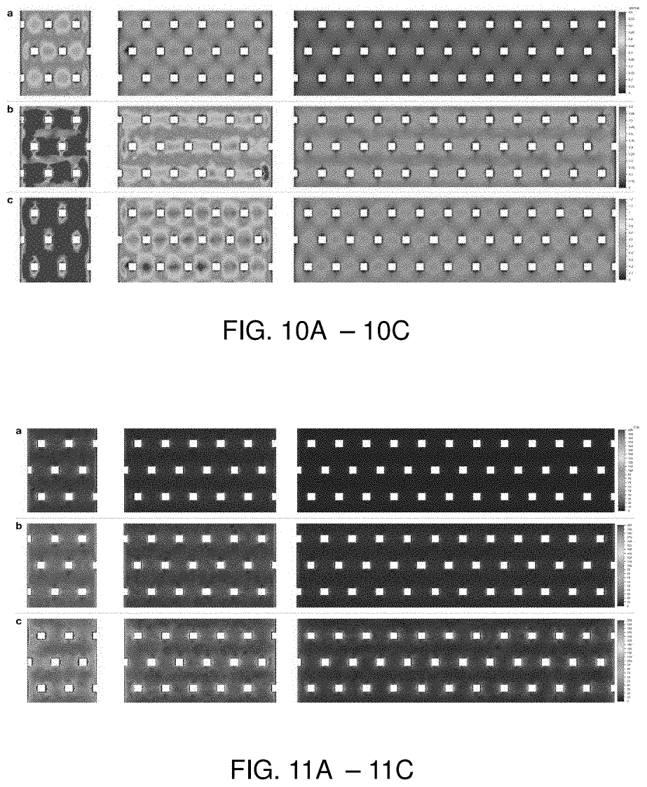

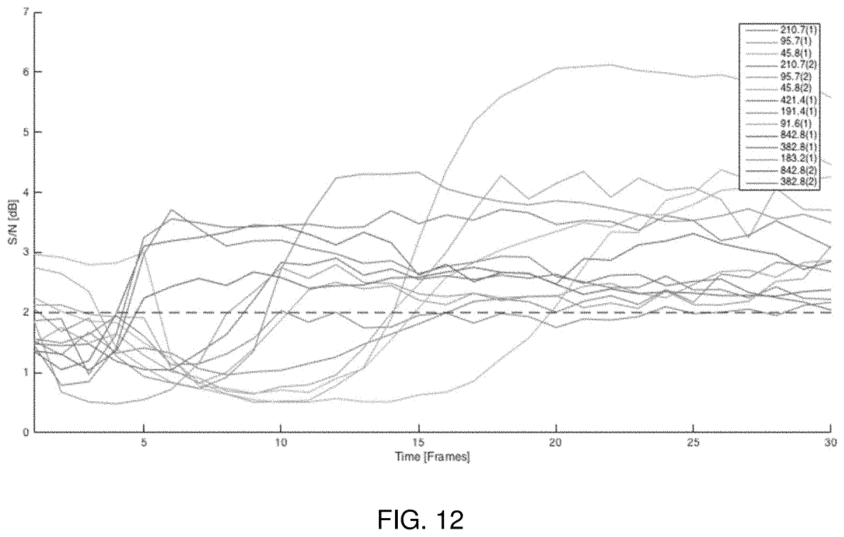

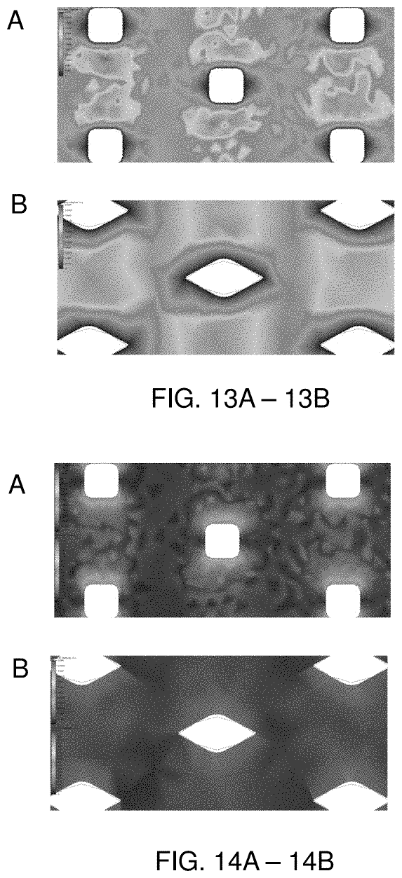

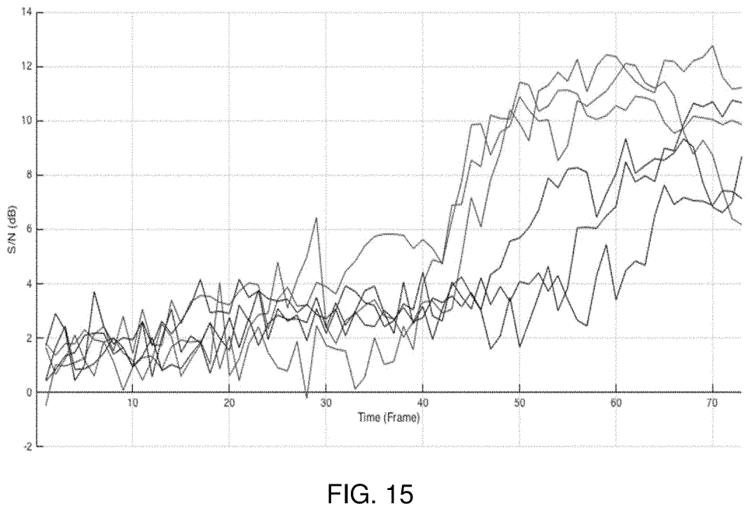

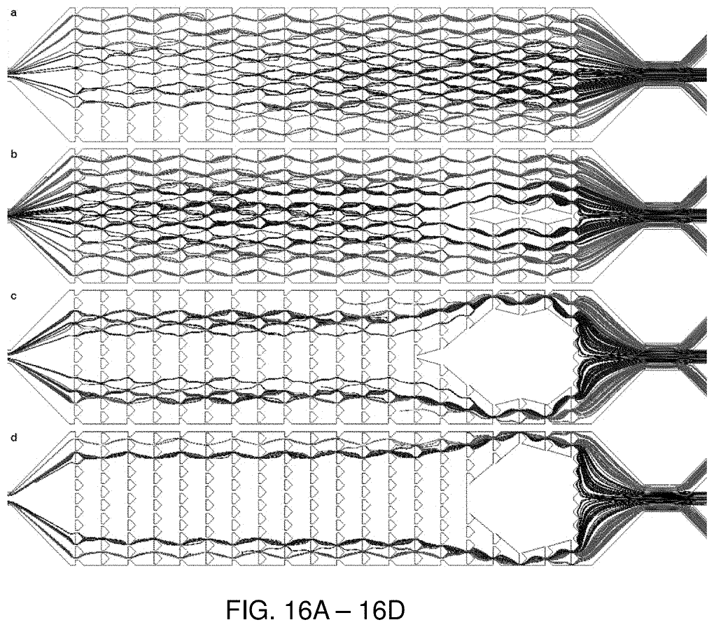

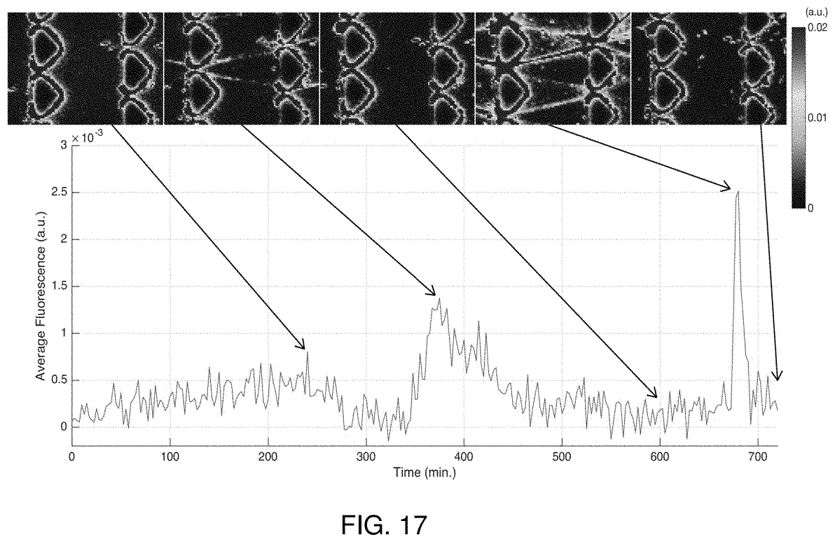

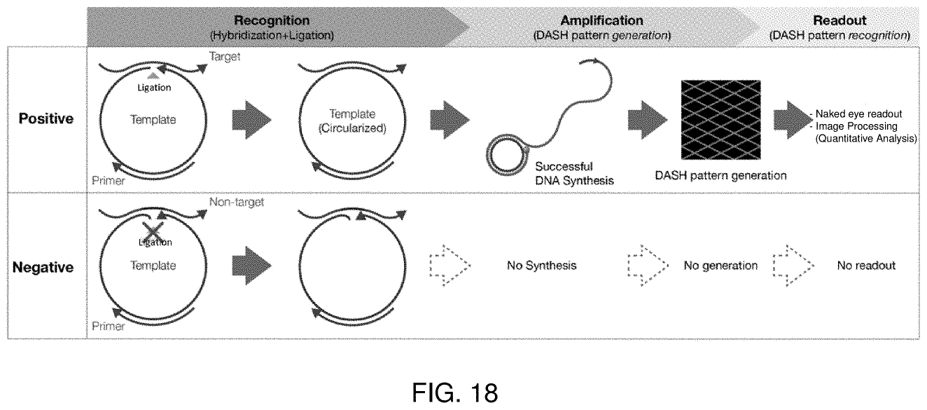

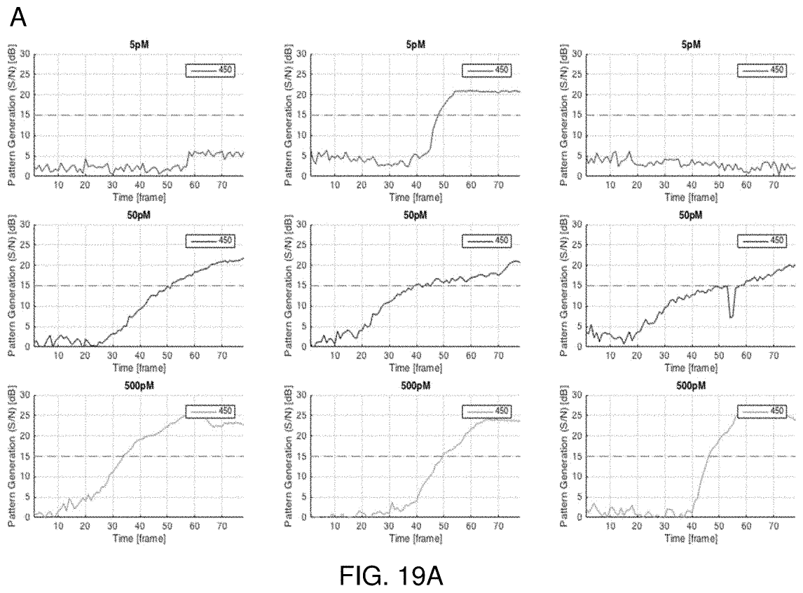

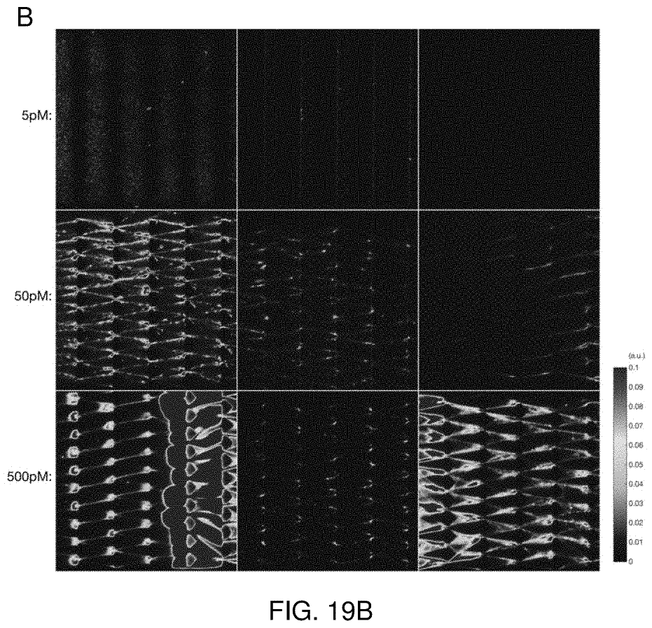

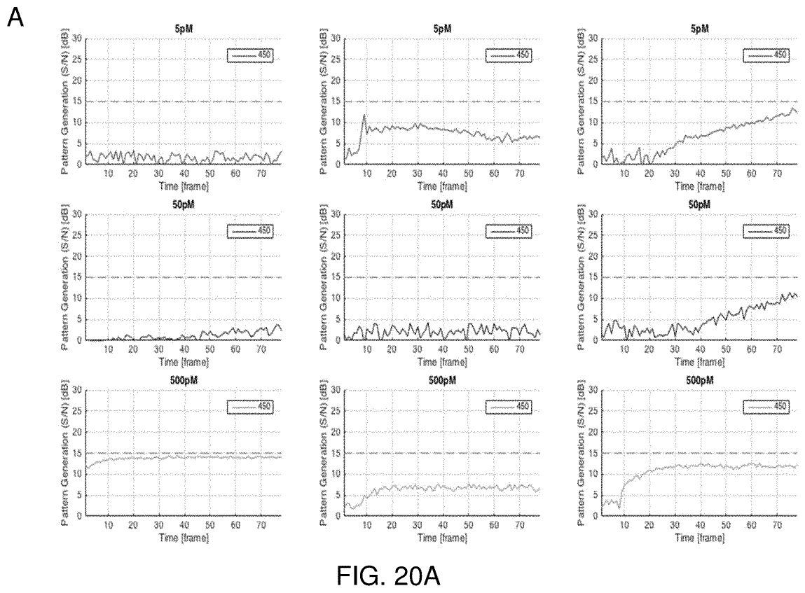

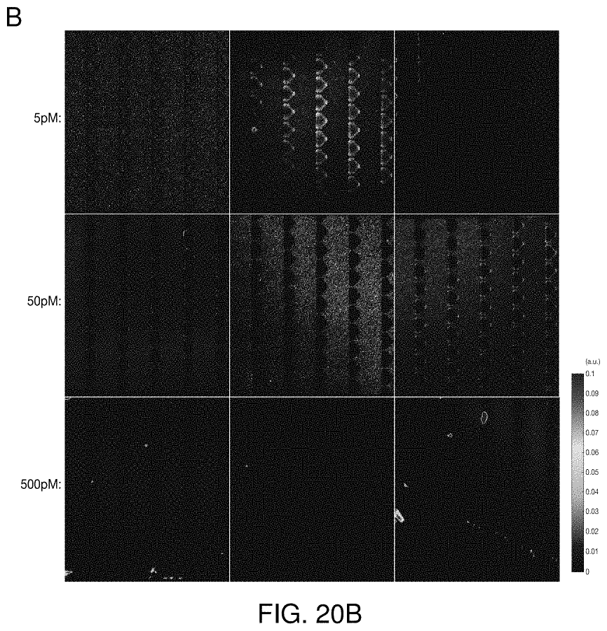

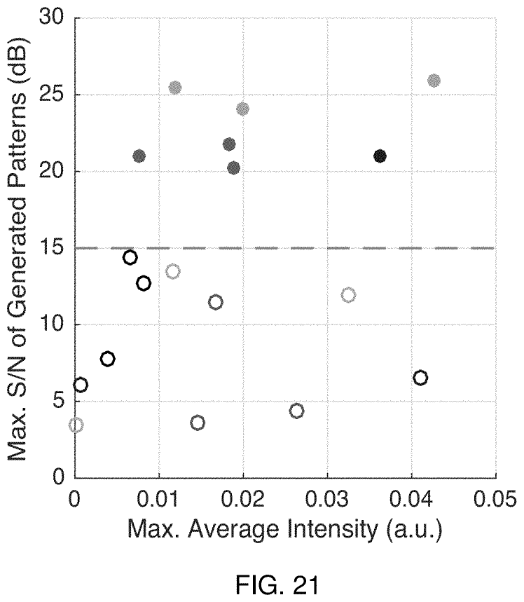

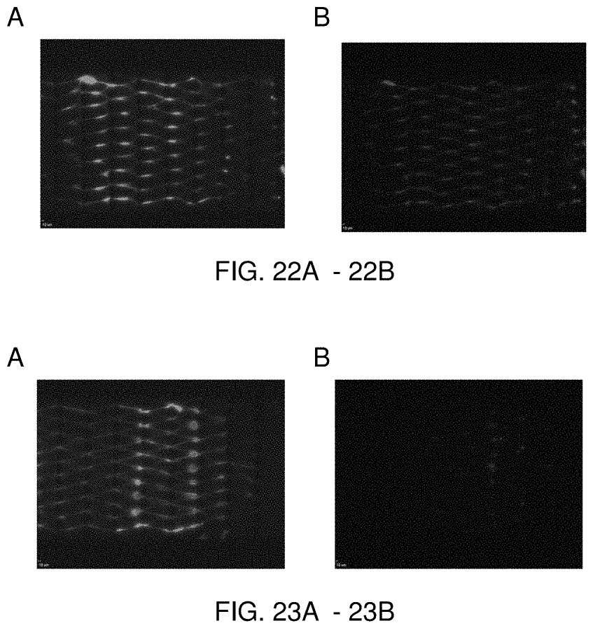

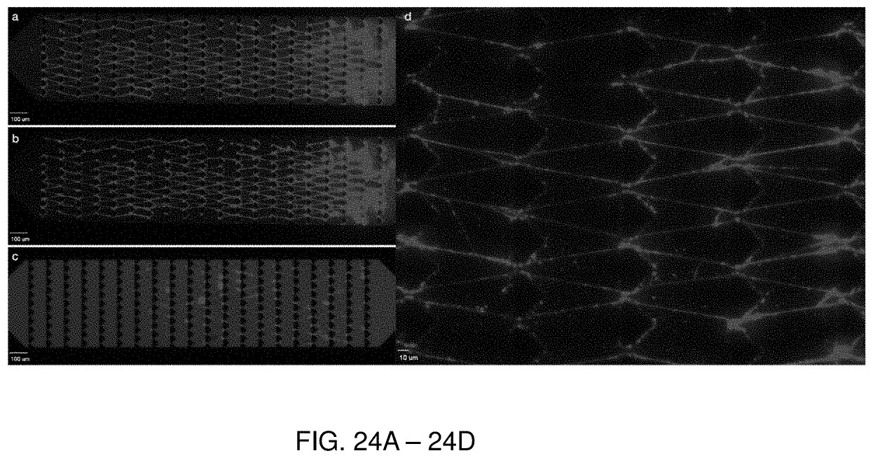

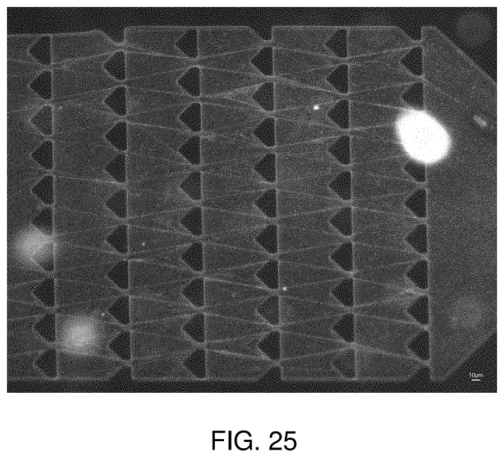

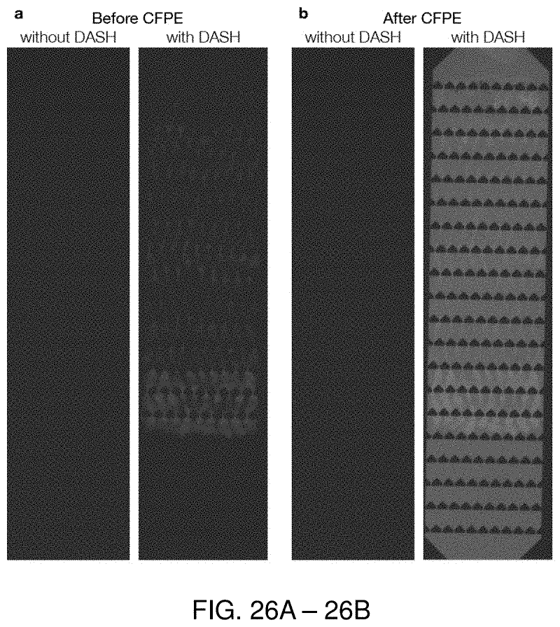

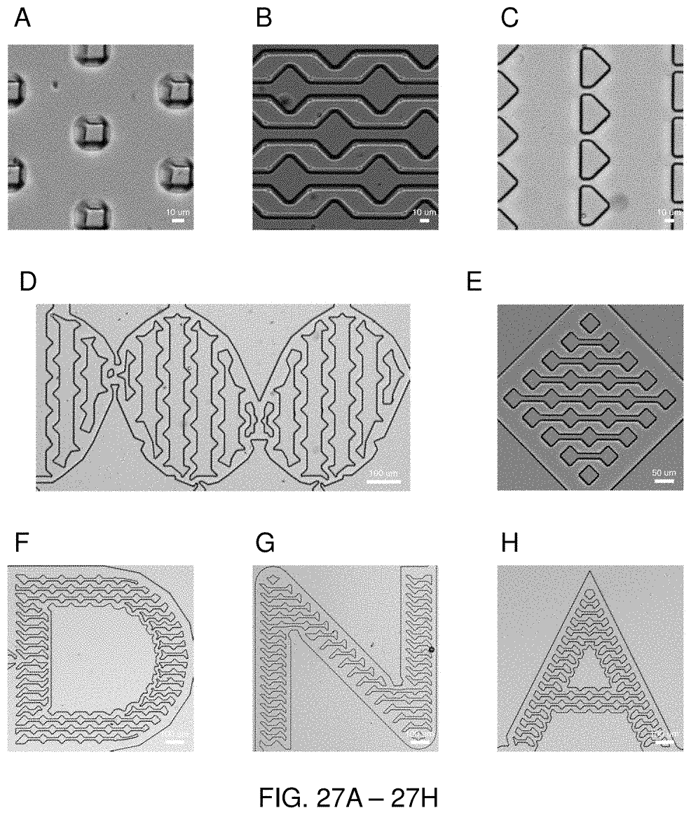

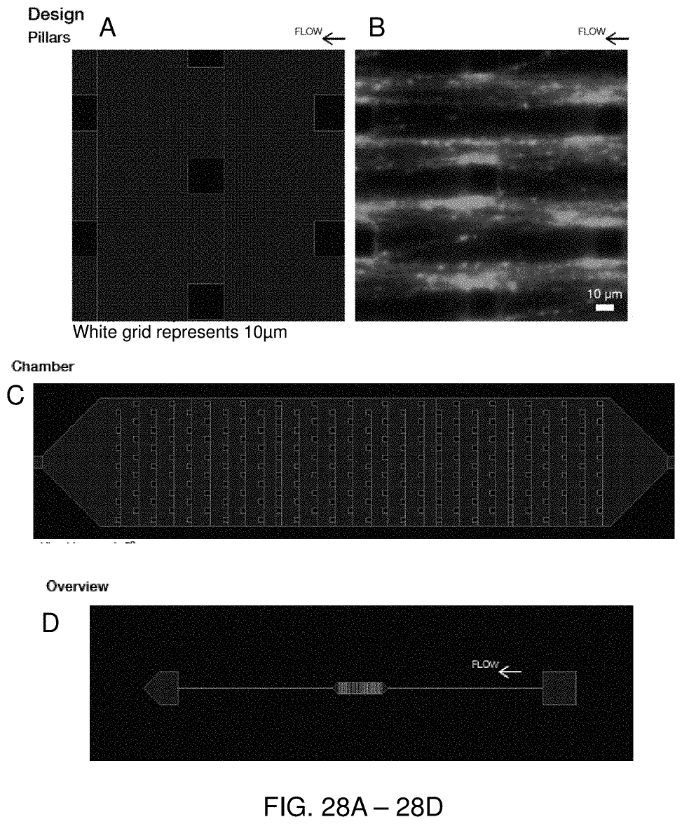

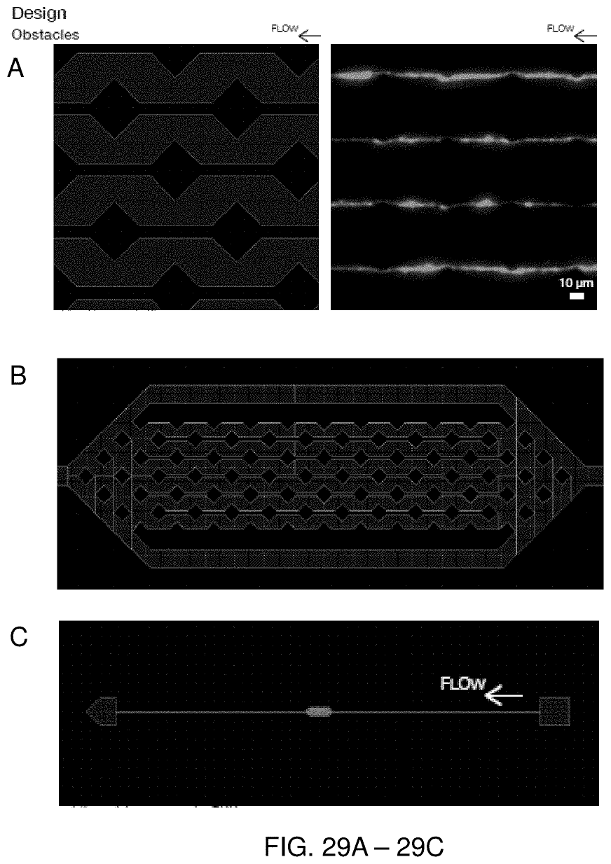

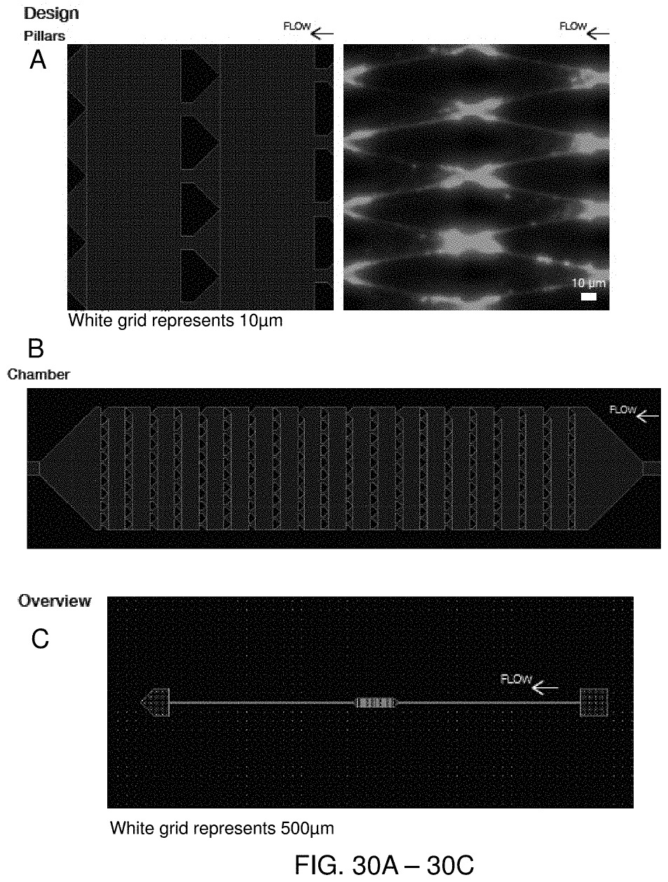

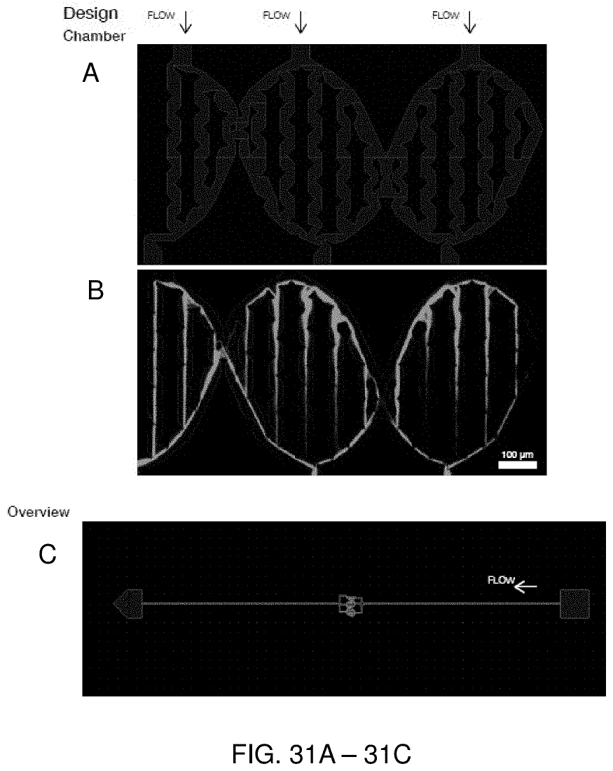

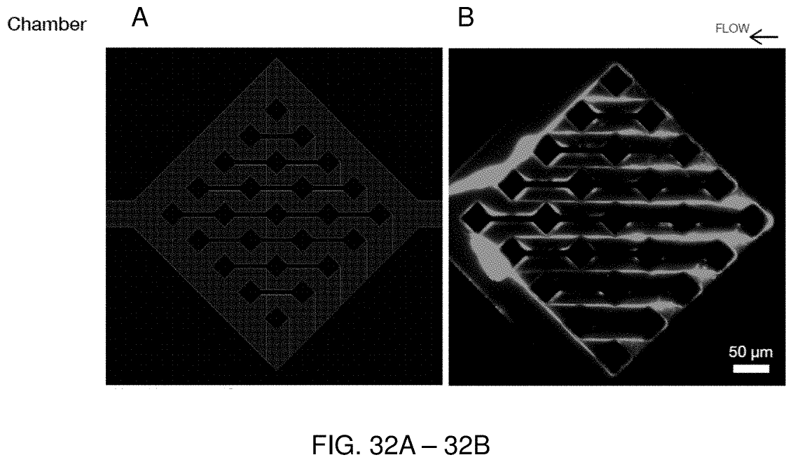

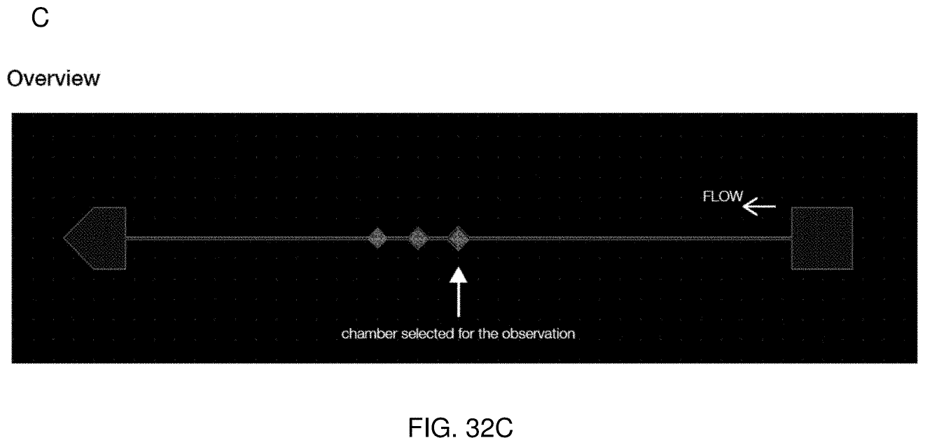

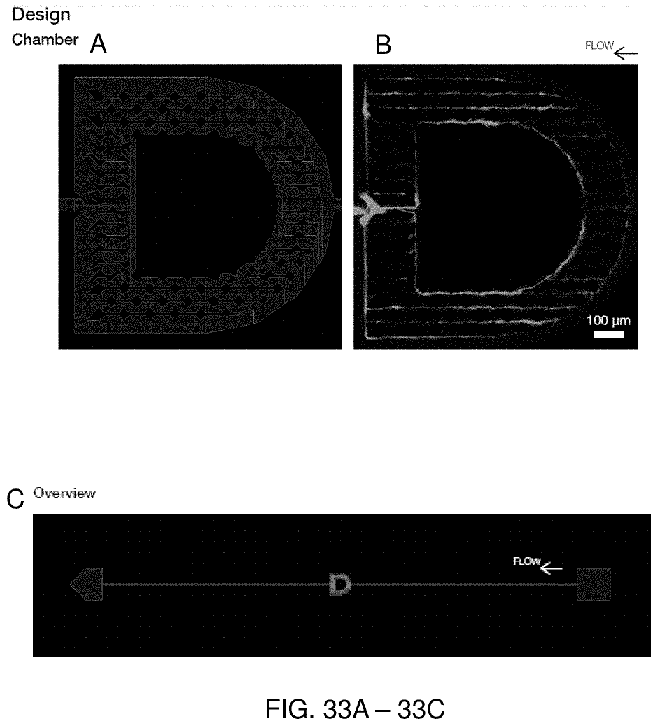

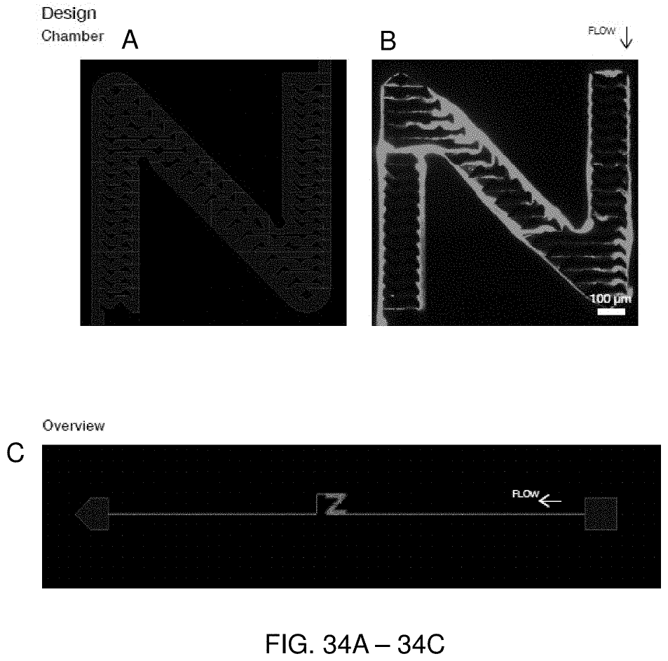

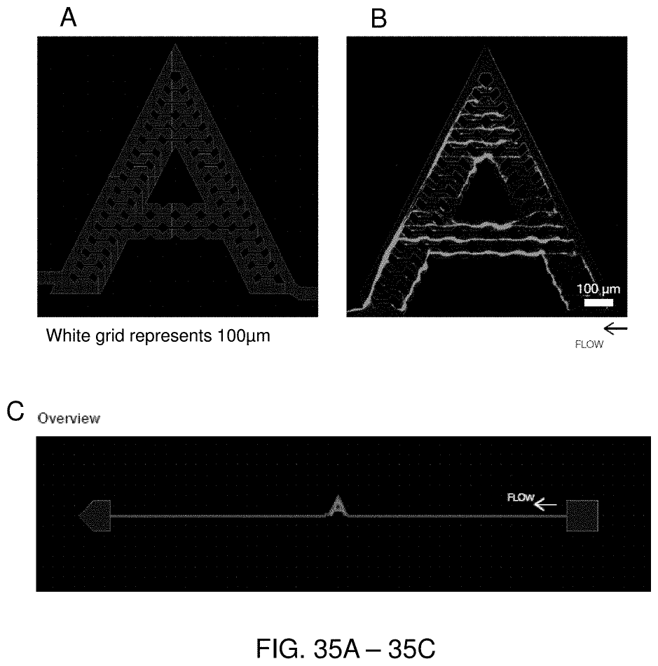

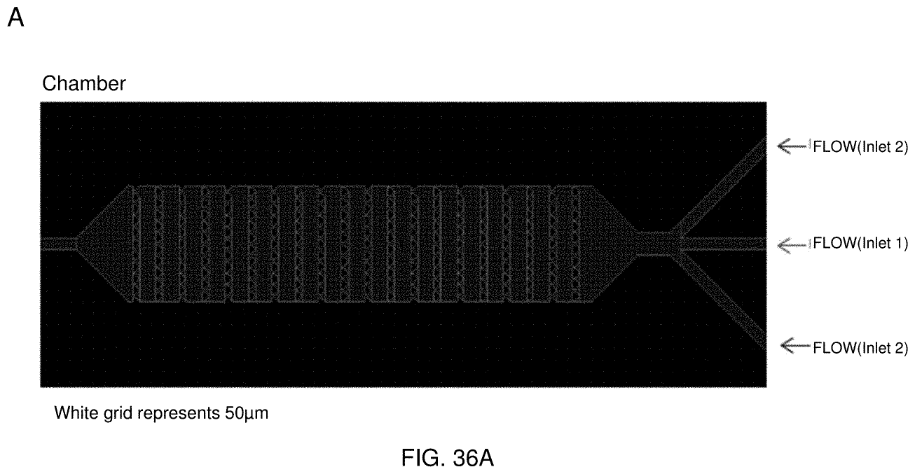

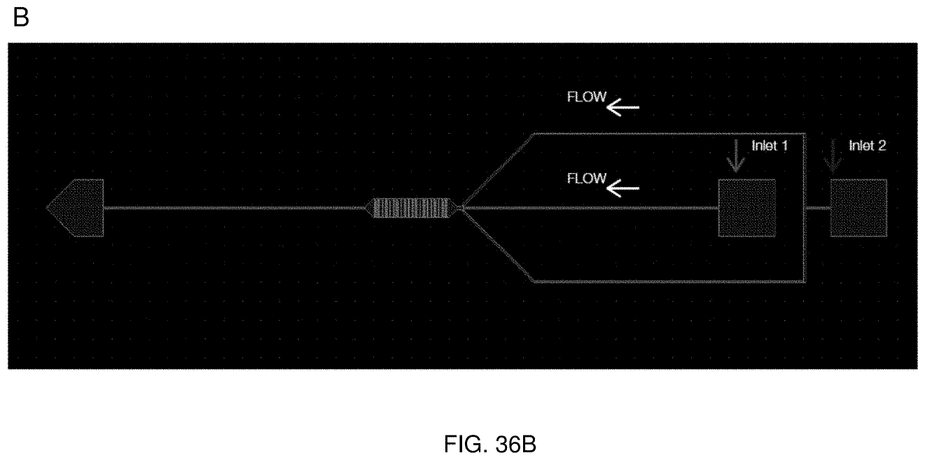

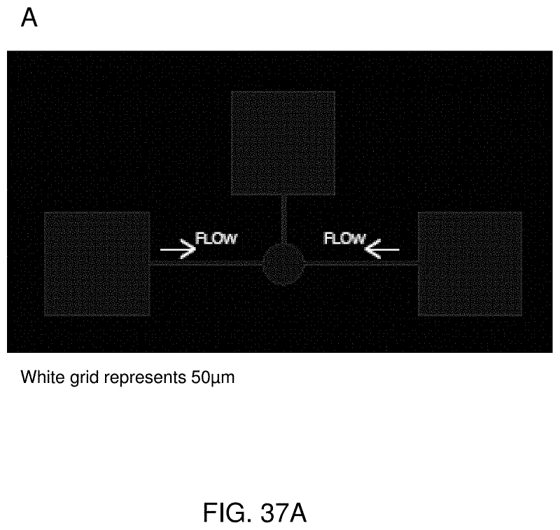

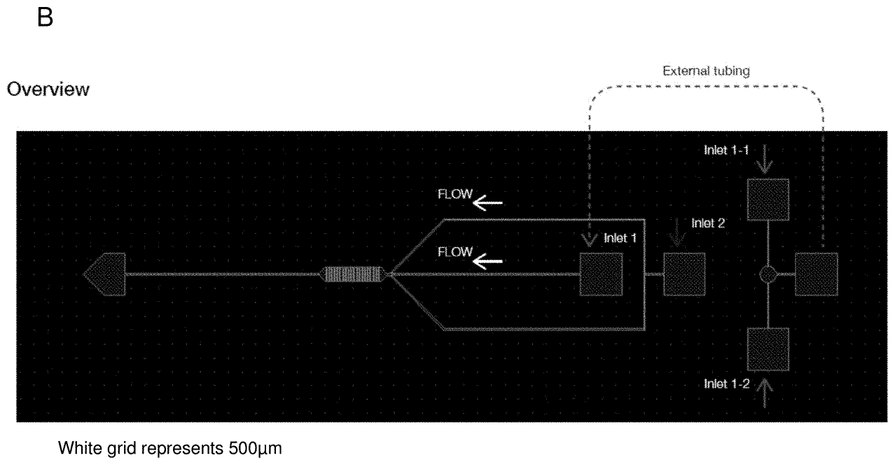

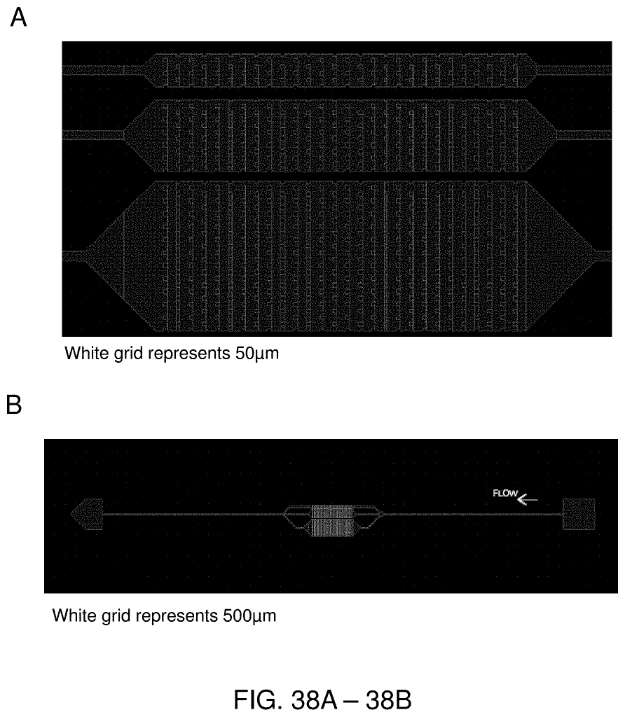

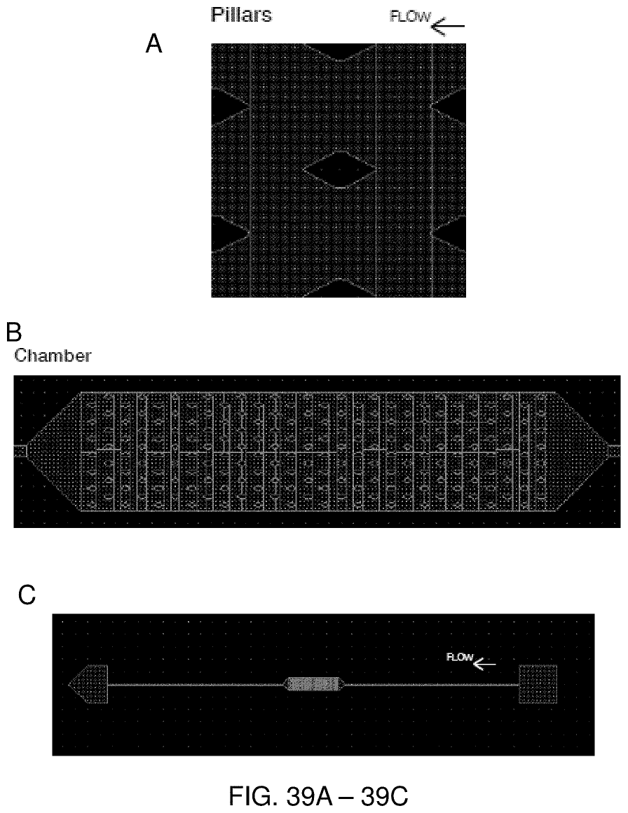

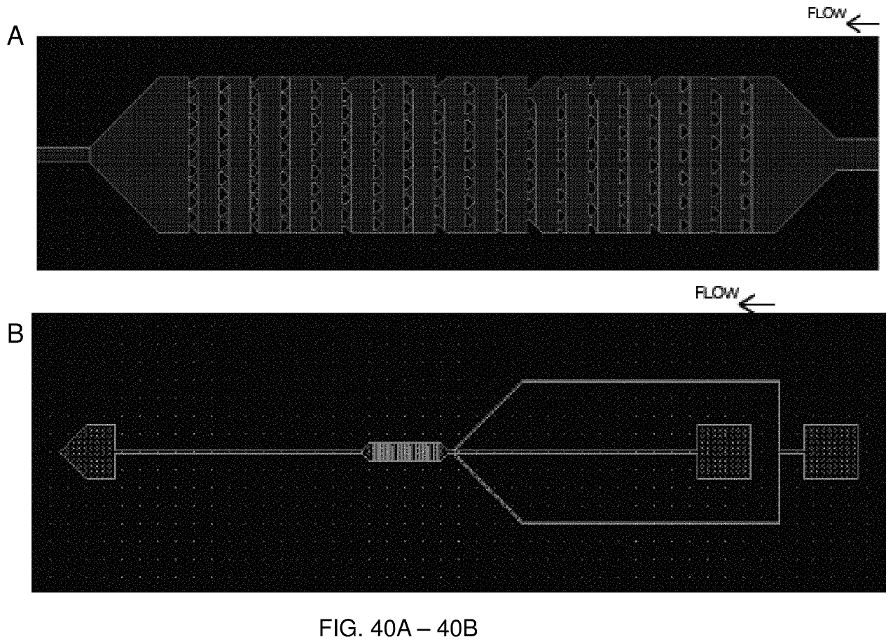

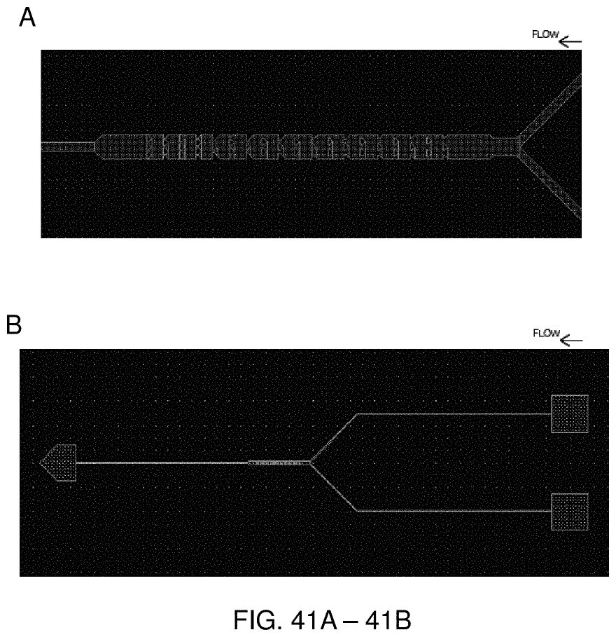

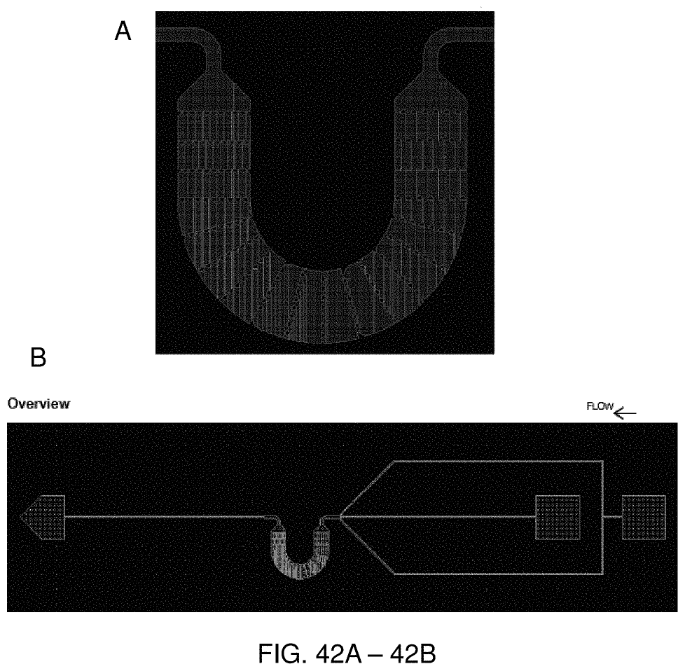

Finally, in addition to using the DASH material in machine applications, several other applications were developed. One of the applications was nucleic acid detection (FIG. 18). The goal of this application was to demonstrate the advantages of the material's self-generating characteristics. A generation seed that was amplifiable when and only when a target pathogen DNA/RNA sequence was present in the sample was prepared. The anabolic characteristic of the material was thus converted to act as a selective amplification process only for targeted DNA/RNA. The generated DASH patterns were then read either by naked-eye observation or by a pattern recognition algorithm based on Fourier transform, employing the mechanism as a binary readout method (FIG. 9). Experimentally, a target sequence taken from Cucumber Mosaic Virus (CMV) was chosen as a model pathogen. The target was successfully detected at 500 and 50 pM concentrations by recognizing the self-generated DASH patterns (FIG. 4A, FIGS. 19A-19B, FIGS. 20A-20B, and FIG. 21). Control targets with a mismatch of only 2 by did not generate the patterns, demonstrating the specificity of the detection method. Next, to illustrate the potential uses of self-generated materials, various hybrid functional materials were created from DASH patterns. The DASH patterns served as a versatile mesoscale scaffold for a diverse range of functional nanomaterials beyond DNA, ranging from proteins to inorganic nanoparticles, such as avidin (FIG. 4B, FIGS. 22A-22B, FIGS. 23A-23B), Quantum Dots (FIG. 4C, FIGS. 24A-24D), and DNA-conjugated gold nanoparticles (FIG. 4D, FIG. 25). The generated patterns were also rendered functional with catalytic activity when conjugated with enzymes. It was also shown that the DNA molecules within the DASH patterns retained the DNA's genetic properties and that in a cell-free fashion, the materials themselves successfully produced Green Fluorescent Proteins (GFP) by incorporating a reporter gene for sfGEP (FIG. 4E, FIGS. 26A-26B). The protein production capability of the materials established the foundation for future cell-free production of proteins including enzymes in a spatiotemporally-controlled manner.

In conclusion, this disclosure is directed to a dynamic material powered by artificial metabolism using simultaneous processes of biochemical synthesis and dissipative assembly. The implementation of the concept, DASH, successfully demonstrated various applications of the material. Notably, the inventors succeeded in constructing machines from this novel dynamic biomaterial with emergent regeneration, locomotion, and racing behaviors, by programming them as a series of FSA. Bottom-up design based on bioengineering foundations without restrictions of life fundamentally allowed these active and programmable behaviors. This material can be integrated as a locomotive element in biomolecular machines and robots. DASH patterns can be easily recognized by naked eyes or smartphones, which leads to better detection technologies that are more feasible in point-of-care settings. DASH can be also used as a template for other materials, for example, to create dynamic waves of protein expression or nanoparticle assemblies.

### Example 2

### Materials and Methods

### Materials

RepliPHI™ Phi29 DNA Polymerase, 10× RepliPHI™ buffer (400 mM Tris-HCl (pH 7.5), 500 mM KCl, 100 mM MgCl₂, 50 mM (NH₄)₂SO₄, and 40 mM DTT) and deoxynucleotides (dNTPs) were obtained from Epicentre (Madison, Wisc.). T4 DNA ligase. Exonuclease I, and Exonuclease III were obtained from New England Biolabs (Ipswich, Mass.). Adenosine Triphosphate (ATP) was obtained from Teknova (Hollister, Calif.). Oligonucleotides were chemically synthesized and purified using standard desalting method by Integrated DNA Technologies (IDT) (Coralville, Iowa). GelRed'M Nucleic Acid Gel Stain and Nuclease free water were obtained from VWR (Radnor, Pa.). SYBR Green I, 40% Acrylamide/Bis (19:1), Ammonium Persulfate (APS), and Polydimethylsiloxane (PDMS) silicone elastomer kit (Sylgard 184, Dow Coming) were obtained from Thermo Fisher Scientific (Waltham, Mass.). Tetramethylethylene Diamine (TEMED) was obtained from Sigma-Aldrich (St. Louis, Mo.).

### Generation Mix Preparation

Generation seeds were prepared by circularizing Template DNA with Primer DNA (FIG. 5). First, chemically synthesized Template and Primer DNAs were mixed in final 1× RepliPHI reaction buffer at equimolar concentration of final 1 μM, then annealed from 95° C. to 4° C. (−1° C./min.) by thermal cycler. 200 U of T4 DNA Ligase and ATP (final 1.25 mM) were added, then incubated overnight at 4° C. (total 20 μL scale, final seed concentration of 0.5 μM) for the reaction. Ligated generation seed solution with final concentration of 5 nM (or otherwise mentioned) were then mixed on ice with final 1 mM each of dNTP, final 1× concentration of SYBR Green I, and 5.7 U/μL of Phi29 in final 1× RepliPHI reaction buffer for the generation mix.

### Microfluidic Device Design

The devices were designed by following three steps. First, a layout of the final DASH patterns was roughly determined. Next, obstacles were assigned by following the patterns using an abstracted method based on node-link diagrams. Total 7 types of standard structural units were used for the design. Finally, the main chamber design was connected to inlet/outlet channels.

All devices were designed by LayoutEditor (Juspertor GmbH, Germany) and KLayout (the Klayout website), and exported to GDSII format. Chrome photomask fabrications were performed by external vendor (Suzhou Mask-Fab Corp., China), except for the initial trials at Cornell NanoScale Science and Technology Facility (CNF) (Ithaca, N.Y.). In CNF, Heidelberg DWL2000 was used for the mask writings; Hamatech-Steag Mask Processor for the development and post-treatment.

Glass wafers (diameter of 4 inches) were washed by water and then immersed into acetone with sonication for 5 min. Then, they were transferred into isopropanol for another 5 min with sonication. After that, the wafers were washed with deionized water and dried in clean air flow. All glass wafers were pretreated by hexamethyldisilazane before photoresist coating. AZ P4620 photoresist (MicroChemicals GmbH, Germany) was dipped onto the wafer center and spun on spin-coater at 1000 r.p.m. for 2 min. to achieve approximately 16 μm thickness. Then, the wafers were baked on hot plate at 95° C. for 8 min and gradually cool down to room temperature. The coated wafer was exposed to UV light on MA/BA6 mask and bond aligner (SUSS MicroTec, Germany) for 30 sec. with quartz mask and then placed into developer composed of az 400K and deionized water with ratio of 1:3 for 2 min. The developed wafers were rinsed with deionized water and dried by air blow. Finally, the wafers were baked on a hotplate at 100° C. for 30 min. to improve photoresist adhesion. Glass wafers were placed on a petri dish (Greiner Bio-One, Austria), and fixed by taping four sides of the edges for the molding process. Microfluidic devices were molded with polydimethylsiloxane (PDMS) silicone elastomer at Base Curing Agent ratio of 10:1 (Sylgard 184, Dow Corning, Corning, N.Y.). After baking at 70° C. for 1 hour, individual devices were cut out from the petri dish, then inlet and outlet ports were punched out. Finally, devices were covalently bonded to PDMS-coated glass microscope slides (MR, Radnor, Pa.) via oxygen plasma treatment.

### Design Process of the Devices

Two empirical guidelines were found based on experimental results and CFD simulations: patterns were 1) formed by following the direction of flow inside the device, and 2) took the shortest route inside the channel, connecting between pillars. Based on these guidelines, the devices were designed by the following deterministic method:

### Device Layout

First, the size of overall devices including channels between inlet/outlets and main chamber were designed. In this paper, channel lengths were set to avoid interference with the objective lens of fluorescence microscope (BX51, Olympus, Japan) when connected to the tubing; typical main chamber length (2 mm) was set based on the image size of the microscope. Channel width between main chamber and inlet/outlets were fixed as 50 μm; typical main chamber width (except complicated geometries such as “D, N, A” letters and “double helix” drawing, 3-chamber device for vorticity control experiments, and the narrow straight track for the locomotion) were set to 500 μm throughout the design for consistency (FIG. 6). Overall size of the device is restricted by the size of glass wafers (7 cm square) by including additional margins for cutting out individual devices and to tightly seal the device during the fabrication process.

### Alain Chamber Design

The layout of main chamber was designed by following three steps. First, the layout of the final DASH patterns was roughly decided. Next, obstacles were assigned by following lines drawn by the first step. Finally, obstacles were merged with channels and main chamber design.

Obstacles were designed based on a combination of boundaries and/or pillars. Total 7 types of standard structure elements were used for the design (FIGS. 7A-7G). Structural elements were categorized into three classes, such as “straight”, “divide” and “merge,” depending on the morphological characteristics of patterns abstracted with node-link diagram. Links represent the final redistributed morphology of DASH structures, and nodes represent the points that DASH structures were generated. The basic geometries were based on solid boundaries with triangular obstacles (FIGS. 7A, 7B, 7F, and 7G). Solid boundaries (gray areas in FIG. 7) define the overall laminar flow direction in the device (blue lines in FIG. 7), DASH structure takes the shortest straight route between top of obstacles (points p, q), so the straight line connecting two points (green lines in FIG. 7) were generated. Typically, channel width was set wider than 20 μm, due to the limitation in the fabrication process. In case of “divide” and “merge” layouts, flow direction defines the overall design of side channels. Since the flow redistributes the generated DASH structure along with the flow direction, the inner corner of the curvature at the branch (points r, u) always needs to be sharp-angled so that the corner becomes generation and anchoring points (i.e., DASH structures were merged/divided at that exact location). The angle between adjacent generation points (between r-s, t-u) defines the angle of generated branch structure. In addition, rectangular obstacles can also be used instead of triangle obstacles (FIG. 7B). Furthermore, in addition to physical boundaries, we can expand this strategy to “imaginary” boundaries by utilizing symmetry of laminar flow inside the device (FIGS. 7C, 7D, and 7E). Once we design pillar structures with line symmetry (blue dashed line), laminar flow also becomes axisymmetric (requires CFD simulation to confirm the symmetrical flow); as a result, we can basically eliminate boundary structures and greatly simplify the design of obstacles by pillars. In this paper, three types of elements with imaginary boundaries were used: zero lateral distance between pillars (c), positive distance (+x) (d), and negative distance (−x) (e). Positive distance defines the maximum width of DASH structure (FIG. 1D); zero (FIG. 1E) and negative (FIG. 1F) distance allows generation of DASH with minimum width. Note that in case of axisymmetric design such as ““letter D”,” the design process can be reduced by simply duplicating most of the upper half of the geometry to the lower half.

Finally, obstacles were merged with channels and main chamber design. The whole process was repeated to refine the patterns by checking the actual DASH pattern formation or CFD simulation results. After iteration, the final optimized design was determined and tested with actual DASH generation experiments.

### Device Design Catalog

Various types of DASH devices and tracks were designed for the generation of patterns by using the methods described (FIG. 27 and FIGS. 28-42). Total 15 types of designs were used in this paper. The catalog summarizes the design of pillars/obstacles, overall geometry of the device/track, and features.

### Sectional Height Measurement of DASH Devices

Height of the chamber was confirmed by sampling actual PDMS devices cut by the blade. Total 42 sections were measured for the analysis. The result shows the average height of 17.4 μm with standard deviation of 1.1 μm, which is in a reasonable range (approximately 8% difference) compared to the ideal thickness of 16 μm.

### Experimental Setup of the Devices

Simultaneous synthesis and assembly using microflow were embodied by a combination of microfluidic device connected to tubing and syringe (FIG. 8), Prepared generation mix solution was drawn into Cole-Parmer Microbore Puri-Flex Autoanalysis Tubing (Vernon Hills, Ill.) connected to lmL BD Medical Tuberculin Syringe (Franklin Lakes, N.J.) and short Microgroup hypodermic tubing (Medway, Mass.) as an insertion tip. Immediately after preparing the solution, the syringe was then set to Harvard. Apparatus PHD-2000 syringe pump (Holliston, Mass.) and infused. Prior to the experiment, DASH devices were prefilled by nuclease-free water; both inlets and outlets were also covered by water. Once the generation mix emerged at the tip, then the tip is immediately inserted to the DASH device. The device and the tip were both covered by solution to make sure that no air bubbles were entering the device during the process. Typically, generation mix was infused to the DASH device at 0.10 μ/min.

Three-inlet design (FIG. 36, #14-2) was designed for generation-degeneration experiments. Center inlet was connected to the generation solution (final seed concentration of 0.1 nM). Side inlets were connected to degeneration solution (DNase (1 U/μL) in final 1× Phi29 reaction buffer). Both generation and degeneration solutions were infused at 0.1 μL/min, For the emergent locomotion experiments, two and three-inlet tracks with gradient vorticity regions (FIGS. 40, 41, 42, #22-3, 23-3, 23-4) were used. Both solutions were infused at 0.15 μL/min. For the emergent racing experiments, three-inlet tracks with gradient vorticity regions (FIG. 41, #23-3) were used. Both solutions were infused at 0.15 μL/min.

### Fluorescence Microscopy

Fluorescence microscopy images used for morphological studies and for quantitative analyses were taken by Olympus BX-61 microscope (Japan) with Sutter Instrument Lambda LS Xenon light source (Novato, Calif.). Green fluorescence (Ex. 484 nm, Em. 520 nm), Red fluorescence (Ex. 555 nm, Em. 605 nm), and Red quantum dot (Ex. 420 nm, Em. 605 nm) filters were purchased from Chrotna Technology Corporation (Bellows Falls, VT), 4× and 10× objective lenses by Olympus (Tokyo, Japan) were used. Exposure time of the bright field channel was set to 100 ms; fluorescence channels were set to 2000 ms throughout all experiments. Time-lapse videos were taken using 150 sec./frame (except the short observation interval video (15 sec/frame) in Supplementary Movie 56) with 4× objective lens. Images including raw data were captured by Intelligent Imaging Innovations SlideBook (Denver, Col.). Raw data (16-bit tiff files) were imported and processed by in-house software for detailed observation.

Confocal laser scanning microscopy (CLSM) images and the z-stacked videos were taken by two confocal laser scanning microscopes (ZEISS LSM710 (Germany), Olympus IX-81 (Japan)). For the time-lapse video recording (Supplementary Movie S2), Device #9-1(FIG. 30) was chosen with final 5 nM of generation mix. 10× objective lens was chosen for the observation due to focal length. Ex. 488 nm, Em. 520 nm filter was used for green fluorescence channel. Capture interval was set to 110 sec; total 30 frames were recorded, 30 layers (z-axis) were taken for each stack.

### SEM

After the generation of the DASH patterns, 4% paraformaldehyde fixative (Electron Microscopy Science, Hatfield, Pa.) was flown into the device (0.1 μL/min) for 10 min. After fixation for 24 hr. at 4° C., the device was opened, and the pattern was fixed on the PDMS substrate. Upon rinsing with nuclear-free water, the pattern was dehydrated in a series of graded ethanol (10%, 25%, 50%, 75%, 90% and 100%) and immersing in 100% ethanol. Subsequently, the pattern was dried with critical point drying process using Baltec (Leica) CPD 408 (Germany) and then examined with LEO (Zeiss) 1550 FESEM (Germany).

### CFD Simulation

2-D CAD files were exported to DXF format from the original CAD design (GDSII), then imported to Rhinoceros 3D (Robert McNeel & Associates, Seattle, Wash.), and simulated using Autodesk Simulation CFD (San Rafael, Calif.). Within Rhinoceros 3D, the original 2-D CAD file was extruded into a 3-dimensional volume with the height that corresponds to the actual DASH devices. The model was then exported as a STEP file in order to be imported in the preparation software within the CFD software. For the fluid flowing through the geometry, the default water profile was used for simplification. For the solid structures, the properties similar to the existing default material of Silicone Rubber were applied. The simulations were then run for maximum 500 iterations or until the result reaches convergence (automatically detected and halted by Autodesk CFD software). Three methods were used for the result visualization of the simulations: heat maps, vector fields, and particle traces. Heat maps and vector fields were normalized between all results to maintain uniformity and were located at 8 μm from the bottom of the volume (mid-point).

### CFD Simulation (Detailed Protocol)

### Introduction

To find small scale trends of the flow through the various geometric patterns, simple Computational Fluid Dynamics (CFD) models were build and run. The aim of this simulation is to establish a simple pipeline (with many generalizations) to estimate the behavior of the flow inside DASH devices, This information was then used as an assistance for the design of new DASH devices, and for an estimation of generation mechanism. Note that the scope of this simulation is at microscale, observing overall behavior of the flow inside the device; further estimation of detailed behaviors, such as nanoscale behavior of flow and polymers including entanglements and network formations, was not considered in this simulation.

### Geometry Preparation

2-D CAD files were exported to DXF format from the original CAD design (GDSII), then imported to Rhinoceros 3D (Robert McNeel &. Associates, Seattle, Wash.), and simulated using Autodesk Simulation CFD (San Rafael, Calif.). Within Rhinoceros 3D, the original 2-D CAD file was extruded into a 3-dimensional volume with the height that corresponds to the actual DASH devices. Some simplifications were made in this 3-D model compared to the actual physical devices, including corners which physically were slightly rounded due to the fabrication process, but in the model remained square as designed. In a similar manner, any rounding at “pillar walls” in the actual physical DASH devices that occurred due to the device fabrication process was designed as straight walls. The inlet/outlet channels of the model were extended as was the case with the physical devices, but to a lesser extent. The extension of these channels would offer very little change in the flow behavior and only increase the mesh count and therefore computing time for each iteration.

### File Transfer and Setup

The Rhino model was then exported as a STEP file in order to be imported in the preparation software within the CFD software. Within the CFD the materials were applied. For the fluid flowing through the geometry, the default water profile was used for simplification. For the solid structures, the following properties similar to the existing default material of Silicone Rubber were applied. While these materials do not have exactly the same material properties as experimental, they were acceptable approximations for the instant purposes.

### Simulation

The simulations were then run for maximum 500 iterations or until the result reaches convergence (automatically detected and hafted by Autodesk CFD software), Three methods were used for the result visualization of the simulations: heat maps, vector fields, and particle traces. Heat maps and vector fields were normalized between all results to maintain uniformity and were located at 8 μm from the bottom of the volume (mid-point). The particle trace was done using particles of 13.8 μm radius and a density of 1.34 g/cm³. These particles were seeded at the inlet face of the geometry.

### DASH Data Import & Analysis Software

Discrete Fourier transform based DASH data analysis software was developed with MATLAB (Natick, Mass.) (FIG. 9), The software uses raw intensity images or videos with multiple channels (Fluorescence channel which contains the DASH patterns, and Brightfield channel which contains overall device outlines) captured by fluorescence microscope as inputs, and quantitatively converts and analyzes the “strength” of the DASH patterns appeared in the images by Fast Fourier Transform (FFT). Note that although the software was mainly used for quantitative measurements of “binary” detection (known as “naked-eye” detection, distinguishing existence/non-existence of patterns) of the DASH patterns for the pathogen detection, the overall process can be readily applied as a general quantitative analysis method of the DASH patterns with 1-D lines or periodical 2-D patterns, regardless of staining methods, types of generation mix, and spatial frequencies.

Here, the overall process used in this paper is briefly described. First, raw images (videos) were imported and pre-processed. The devices were recorded with random positions with random angles in the original raw image, so the first step needs to standardize the data by correcting the rotation and location of the device followed by cropping the image, along with subtracting the background values in the fluorescence channel. The background intensity was subtracted from an average intensity of 100 pixels in the image (picked from an area inside the chamber that does not contain DASH patterns). The process was repeated for all frames in the video. In some cases, due to its elastic property of the PDMS-based device, the device itself slowly moved during the observation. In those cases, an additional image stabilization process was applied before the preprocess to ensure consistency throughout all frames. The imported images and videos were used throughout the paper.

Next, FFT was applied to the images, frame-by-frame. In case of CMV pathogen detection, zig-zag geometry (FIG. 30, #9-1) with crosshatch patterns was used for the experiment, so 2-D FFT was selected as a conversion method. Square region (310px×310 px, equivalent to 500 μm×500 μm in the original size) was selected from the sample image, and converted into frequency domain. The same location was chosen throughout all frames in the video. (In case of 1-D FFT, each strip of row (1 px×310 px) was converted one by one.) After the conversion, spatial frequency peaks that correspond with the DASH patterns were selected. In case of 2-D zig-zag geometry, the fundamental frequency of f=10 (Hz) with corresponding angles were chosen. This process can be interpreted like an “opposite” of typical notch filtering process. Usually, notch filter removes certain peaks in the frequency domain image in order to eliminate spectral periodic noises. However, in this DASH pattern's case, those periodic patterns with specific spectral peaks are the signal, instead of noise (and vise-versa), The strength of this method is that once we design the DASH patterns, then the spatial frequency and angles of patterns were deterministically defined without any arbitrary parameter for tuning. This method can greatly simplify and ensure the accuracy of overall quantitative analysis. For instance, since this 2-D FFT method can pick specific spatial frequency with specific angles, so the noises with other spectrum and/or angles, such as weak DNA attachment to pillars (including same or similar frequency but with different angles), and random high backgrounds, can be automatically distinguished and counted as noise from the actual DASH patterns (signal). Finally, Signal-to-Noise ratio (SNR) of the DASH pattern signal in the image was calculated (higher the stronger DASH pattern generation) frame-by-frame, and used as a quantitative indicator of the generated patterns (FIG. S33).

### DASH-Based Pathogen Detection

A sequence taken from Cucumber Mosaic Virus (CMV) was used for the target. Total 2 bp mismatch (1 bp at each side of the ligation site) of sequence alternation was made for the non-target. For simplification of the experiment, the total target sequence length was shortened to 33-mer; chemically-synthesized single strand DNA was used instead of RNA. Recognition was performed by adding target DNA to a solution containing template and primer DNA in final 1× RepliPHI Phi29 buffer. Following the annealing process (from 95° C. to room temperature at −1° C./min.), final 10 U/μL of T4 DNA Ligase were added along with 1.19 mM ATP and the reaction left at 4° C. overnight. Generation mix for the amplification (DASH pattern generation) was then prepared by following the standard method with corresponding concentration of ligated template-primer mix; the solution was then infused to the device (FIG. 30, #9-1) at 0.1 μL/min. for maximum of 4 hrs. Time-lapse video was recorded during the process; the results were then imported using in-house software.

### DASH-Avidin/Streptavidin Hybrid Materials

The DASH pattern was generated using the standard protocol with zig-zag pattern device (FIG. 30, #9-1) over 1 hr.-1 hr. 20 minutes. To verify that the DASH pattern had formed correctly, 1× final concentration of SYBR green I was included in the generation mix. Immediately following DASH generation, a 50 μg/mL solution of either Texas Red-conjugated avidin or Texas Red-conjugated streptavidin in 1× RepliPHI reaction buffer was flowed through the device at 0.1 μL/min for 1 hr. Fresh 1× RepliPHI reaction buffer was then flowed through the device for 30 min to remove any unbound protein before imaging.

For two-colored Avidin binding, in order to ensure that the DASH patterns formed evenly throughout the entire device, DASH generation solution was pumped through all inlets simultaneously (each at 0.1 μL/min). To avoid spectral overlap with FITC-conjugated. Avidin, SYBR Green I was not included in the generation mix, After DASH formation, 50 μg/mL of Texas Red-conjugated Avidin and 50 μg/mL of FITC-conjugated Avidin were pumped simultaneously into the device (one Avidin conjugate per inlet) at 0.14/min each for 1 hr. Prior to imaging, the device was flushed with 1× Phi29 reaction buffer for 15-30 min to reduce background.

### DASH-Quantium Dots Hybrid Materials

The DASH pattern was generated using the standard protocol over 1 hr. 30 minutes without SYBR Green I. Immediately following DASH generation, a 250 μg/mL solution of FITC-conjugated avidin (Thermo Fisher Scientific, Waltham, Mass.) in 1× RepliPHI reaction buffer was flowed through the device at 0.1 μL/min for 1 hr., then followed by final 0.2 μM of Biotin-labeled Qdot 605 nanocrystals (Thermo Fisher Scientific, Waltham, Mass.) in 1× RepliPHI reaction buffer for 10 to 30 minutes. Control samples were tested without FITC-conjugated Avidin binding process.

### DASH-AuAP Hybrid Materials

Citrate coated 40 nm and 5 nm gold nanoparticles were purchased from Ted Pella (Redding, Calif.). The oligonucleotides used were ordered conjugated with a 5′ thiol group from Integrated DNA Technologies, which was activated prior to attachment by deprotection using tris(2-carboxyethyl)phosphine hydrochloride (TCEP). The oligonucleotides were incubated at a ratio of one to five (DNA:TCEP). The deprotected DNA was then added to the AuNPs at a DNA:AuNP ratio of 80:1 for the 5 nm and 4200:1 for the 40 nm gold nanoparticles to ensure maximum surface coverage, followed by overnight shaking at 500 rpm at room temperature. NaCl was then slowly added over a period of 8 hours to a final concentration of 500 mM, reducing DNA-DNA repulsion and further increasing the DNA coverage. The nanoparticles were then purified of salt and excess DNA by 5 rounds of centrifugation in nuclease free water.

The DASH pattern was generated using the standard protocol using Device #9-1 (FIG. 30). After 70 minutes of generation, DNA-conjugated 5 nm or 40 nm AuNP solution with final 1× RepliPHI buffer was flown inside the device (0.1 μL/min.) for 45 min. The process was constantly monitored by microscope to ensure the sufficient attachment of nanoparticles.

### Cell Free Protein Expression

The DASH pattern generation was performed by following a protocol similar to the standard protocol, with final seed concentration of 15 nM with 8 mM mix of dNTP, 4 U/μL of RepliPHI Phi29 DNA polymerase. and 0.5 μg/mL of Hoechst 33342, in final lx RepliPHI buffer. Blue Hoechst dye was used to stain DNA for the confirmation of DASH pattern generation instead of SYBR Green I in order not to overlap with the green emission wavelength of sfGFP for the subsequent protein expression steps. The generation process was monitored by time-lapse observation until the pattern generation was complete.

After the DASI-I pattern generation process, a protein expression primer was infused at 0.1 μL/min. for 60 min. The primer sequence was designed to bind. T7 promoter regions present on DASH patterns in order to activate protein expression. S30 T7 High-Yield Protein Expression System from Promega (Madison, Wisc.) was then used for the protein expression. Nuclease free water, S30 Premix Plus and S30 T7 Extract (both supplied with kit) were mixed in a 2.4:4:3.6 ratio and infused at 0.1 μL/min. For the direct observation of CFPE in the DASH device, the pump was programmed to pause every 20 minutes to increase the residence time to directly observe the fluorescence in the microfluidic device. For a quantitative measurement (FIG. 4E), solution from a total of 2 hours of CFPE in the device was collected and fluorescence was measured using a Synergy 4 Microplate Reader (with filters of Ex. 475 nm, Em. 508 nm) from BioTek (Winooski, Vt.).

### Sequences

Generation seeds were designed by combination of Template and. Primer DNAs. The following sequences were used throughout all generation and degeneration experiments in this disclosure (except some control experiments, DASH-based detection, and cell-free protein expression experiments)

Primer (T1c):
(SEQ ID NO: 1)
GACCACCTTCGCGTCCAAAGC
Template (T2-Eco):
(SEQ ID NO: 2)
CGAAGGTGGTCTTTTTTTTTAT
ATAGAATTCTATATATTTTTTT
TGCTTTGGACG

Notes: Sequences are written with 5′->3′ direction. 5′ and 3′ pairs represent complementary sequences. Template DNA was prepared with 5′ phosphorylation for the ligation process.

Primer (T1c-NCTRL):
(SEQ ID NO: 3)
CAACCAAACACCCCAACCACC
Template (T2-NCTRL):
(SEQ ID NO: 5)
GTGTTTGGTTGTTTTTTTTTTT
TTTTTTTTTTTTTTTTTTTTTT
TGGTGGTTGGG

A Template sequence is composed of two segments of complementary sequences to the Primer (Blue and Red) with additional center domain connected by linkers using poly-T sequences. Sequences were designed by in-house version of DNADesign MATLAB toolbox running on Eclipse (original MATLAB version was developed by Winfree group (Caltech Centrosome DNAdesign website)). For applications such as pathogen detection and protein expression, different sets of template/primers with specific sequences were designed for generation seeds.

### Generation Seed Ppreparation: Gel Electrophoresis

Gel electrophoresis confirmed 1× and 2× sized circular templates were successfully formed after the reaction. In this case, according to the results, 2× circular templates were also generated due to palindromic sequence in the template, which led to hybridize with another template and ended up with double-sized length. T2-NCTRL and T1c-NCTRL showed only 1× sized circular template formation after ligation.

### Experimental Setup of the Device

Simultaneous synthesis and assembly using microflow were embodied by a combination of microfluidic device connected to tubing and syringe (FIG. 8). Prepared generation mix solution was drawn into Cole-Parmer Microbore Puri-Flex Autoanalysis Tubing (Vernon Hills, IL) connected to 1 mL BD Medical Tuberculin Syringe (Franklin Lakes, NJ) and short Microgroup hypodermic tubing (Medway, Mass.) as an insertion tip. Immediately after preparing the solution, the syringe was then set to Harvard Apparatus PHD-2000 syringe pump (Holliston, Mass.) and infused. Prior to the experiment, DASH devices were prefilled by nuclease-free water both inlets and outlets were also covered by water. Once the generation mix emerged at the tip, then the tip is immediately inserted to the DASI-I device. Note that the device and the tip were both covered by solution to make sure that no air bubbles were entering the device during the process. Typically, generation mix was infused to the DASH device at 0.1 μL/min.

Additional Notes on the Setup of Generation-Degeneration/Locomotion/Racing Experiments

### Generation-Degeneration, Locomotion, and Racing Experiments

Three-inlet design (FIG. 36, #14-2) was designed for generation-degeneration experiments. Center inlet (1: red in #14-2 catalog) is connected to the generation solution (final seed concentration of 0.1 nM). Side inlets (2: blue, divided into two inlets) are connected to degeneration solution (DNase I (1 U/μl) in final 1× Phi29 reaction buffer). Both generation and degeneration solutions were infused at 0.1 μL/min. For the emergent locomotion experiments, two and three-inlet tracks with gradient vorticity regions (FIGS. 40, 41, 42 #22-3, 23-3, 23-4) were used. Both solutions were infused at 0.15 μL/min. For the emergent racing experiments, three-inlet tracks with gradient vorticity regions (FIG. 41 #23-3) were used. Both solutions were infused at 0.15 μL/min.

### Repeated Generation-Degeneration Experiments

To ensure constant synthesis reaction time, three-inlet device with additional T-junction module (ID#20-2) was designed. External tubing with the length corresponding to 2 hr. synthesis reaction time (under 0.1 flow rate) connected between outlet of T-junction and Inlet I of two-inlet device. Reaction mix solution with Phi29 enzyme but without dNTP was used for Inlet 1-1; without Phi29 but with final 2 mM dNTP was used for inlet 1-2. Both 1-1 and 1-2 were infused at 0.05 μL/min. Solutions were mixed at the center reservoir of T-junction; synthesis occurred during flowing through the external tubing for 2 hr. Same DNase I solution used in generation-degeneration experiments were used for Inlet 2 (infused at 0.1 μ/min.).

### Transfer Methods for SEM Observation

For SEM observation of samples including DASH-AuNP patterns, transferrable DASH devices were prepared by “peelable” PDMS device setup using adhesive tape as a substrate. Note that the overall process can be used as a general transfer technique for future application use of this DASH platform, in addition to SEM sample preparation. 3M Scotch tape (Maplewood, MN) was used as substrate by placing upside down (i.e. adhesion side on top) on a glass slide, and then PDMS chamber device was gently placed on top and sealed. After DASH pattern generation and gold nanoparticle attachments by following typical methods mentioned above, the device was immediately frozen at −80° C. freezer for overnight. PDMS device was then peeled off from the substrate at room temperature before the solution inside chamber starts to melt. As a result, DASH patterns were transferred to the substrate side since the structures were retained inside ice during the removal process.

### Sequences Used in Pathogen Detection

CMV target:
(SEQ ID NO: 5)
CTGAGTGTGACCTAGGCCGGCATCATTGGATGC.
Non-target:
(SEQ ID NO: 6)
CTGAGTGTGACCTAGGAAGGCATCATTGGATGC.
Template:
(SEQ ID NO: 7)
GCCTAGGTCACACTCAGTTTTTTTTCGGTGCGA
GTTTACGCTCTACTTTTTTTTGCATCCAATGATG
CCG.
DASH generation primer:
(SEQ ID NO: 8)
GTAGAGCGTAAACTCGCACCG.

### Sequence Used in DASII-AuNP Hybrid Material Generation

Linker:
/5ThioMC6-D/TTTTTTTTTTTTTT
TTTTTTTTTTTTTTGCTTTGG > 3′

Linker DNA was designed with poly-Ts followed by a segment (shown in italics with the complementary sequence of synthesized. DNA from T2-Eco template, Thiol group modification was added at 5′ side for the conjugation process mentioned above.

### DASH-Enzyme Functionalization Method

The DASH pattern generation was performed by following the standard protocol with final seed concentration of 500 pM. The generation process was monitored by fluorescence microscope for maximum of 4 hr. until the pattern generation was complete. Following the pattern generation, avidin-HRP solution from Bio-rad (Hercules, Calif.) was prepared at concentrations of 10 μg/ml or 100 μg/ml in final Ix RepliPHI buffer with final 1× SYBR Green I, and infused to the device at 0.1 μL/min. for 1 hr. Excess avidin-HRP was then washed off by flowing through a solution of final 1× RepliPHI buffer with final 1× SYBR Green 1 at 0.1 μL/min. for 1 hr. One-Step Ultra TMB-ELISA substrate solution from Thermo Fisher Scientific (Waltham, Mass.) was used for the HRP activity assay bound to the DASH patterns. TMB substrate solution was infused to a device at 0.1 μl/min for 1 hr. For the confirmation of the localization of the HRP activity from the DASH patterns, in situ HRP reaction was monitored by using QuantaRed Enhanced Chemilluorescent HRP Substrate Kit from Thermo Fisher Scientific (Waltham, Mass.). QuantaRed solution of 1 ng/mL or 1 μg/mL was infused to the device with flow rate of 0.1 μL/min. and continuously monitored by fluorescence microscope for maximum of 2 hrs.

### Additional Methods and Sequences Used in Cell-Free Protein Expression

### Generation Seed Preparation

A circular DNA template for DASH generation seed was prepared by using a plasmid containing sfGFP (super-folded Green Fluorescent Protein) sequence. First, 100 ng of plasmid was prepared in a solution of final 1× NEBuffer from New England Biolabs (Ipswich, Mass.) mixed with final 0.25 U/μL of Nb.BsmI Nicking Endonuclease from New England Biolabs (Ipswich, Mass.). The solution was incubated at 65° C. for 5 hours followed by an enzyme inactivation step at 80° C. for 20 minutes and then cooling down to room temperature at −1° C./min. Next, final 0.2 U/μL of Exonuclease I and final 1 U/μL of Exonuclease III were then added and the reaction was incubated at 37° C. for 5 hours. Exonuclease was then inactivated at 80° C. for 20 minutes followed by an annealing process down to room temperature at −1° C./min. The gel band showed a successful formation of circular template DNA from original double stranded plasmid DNA. The solution containing single stranded circular template was then buffer exchanged using a 30 k Amicon Ultra Centrifugal Filter from EMD Millipore (Billerica, Mass.) with 8 μl of nuclease free water per μl of reaction solution and centrifugation at 10,000×g. The addition of water followed by centrifugation was repeated twice before collecting the template. DASH generation primer was then hybridized to the template in a 1:1 molar ratio by annealing the solution at −1° C./min. from 95° C. down to room temperature.

### Sequences

DASH generation primer:
(SEQ ID NO: 10)
CAAAAAACCCCTCAAGACCC
Protein expression primer:
(SEQ ID NO: 11)
TAATACGACTCACTATAGGG

Green fluorescent protein expression template (plasmid) (SEQ ID NO: 12).

### Example 3

### Control Experiments of the DASH Pattern Formation Process

### Redistribution After Pre-Formation of Large (Gel-Like) Networks

Instead of simultaneous synthesis and formation, pre-formed DNA networks were redistributed inside the DASH device for the control. Generation mix (final 0.5 nM) were incubated for 1, 2, 4 hours inside 0.6 mL tube at room temperature, then heated up to 90° C. for 20 min., quickly quenched by ice, and flowed through the device (0.1 μL/min.) for 2 hours. Note that samples were heated to deactivate enzyme reactions and stop additional synthesis during the assembly (device-flow) process, followed by quick quenching to enhance the formation of networks. At equivalent condition (device type, flow rate, seed concentration), DASH structures typically start forming fibrous network structures at around 2.5 hours after the start of reaction. However, all samples created random gel-like aggregations throughout the device with this condition; no DASH patterns were observed. This result suggests redistribution of small networks (and in situ network formation) might be one of the key mechanisms behind DASH formation (i.e., pre-formed large aggregations cannot easily change its morphology into fibrous patterns during redistribution process once they formed).

### Redistribution Without Pre-Formation of Large Gel-Like Networks

Another control experiment was performed in order to clarify the formation process during the DASH generation. Redistribution of the pre-synthesized DNA (T2-NCTRL template, final 5 nM) with optimal timing (2.5 hours) were tested, but this time without enhancing pre-formation of gel-like large aggregations by quenching (i.e. all processes were performed in room temperature). Here, instead of using heat-based deactivation of enzymatic reactions, Proteinase K from New England Biolabs (final 0.02 U/μL) was mixed after 2.5 hours of growth inside 0.6 mL tube. The solution was then run for 4 hours with 0.1 μL/min.

The result shows that pre-synthesized long DNA can be redistributed and can form DASH patterns with this method (in a non-autonomous fashion, based on manual manipulation). However, in this case, since the formation is occurring simply due to the redistribution of synthesized long DNA presumably in a morphology of smaller pre-formed networks, non-uniformed pattern is observed inside the device (i.e., only upstream side (right half of the image) contained fibrous patterns). As mentioned in the main text, continuous and simultaneous synthesis and flow, notably vorticity, is the key to trigger local formation of networks at the side of pillars, and thus leads to uniformed generation of the DASH patterns inside the device.

### Contribution of DNA Hybridization During the Formation Process

Finally, the mechanism behind the network formation is further investigated using redistributions in a similar fashion to the experiment shown above. T2-NCTRL template with final 5 nM concentration was used for the generation mix. Here, after 2.5 hours of synthesis in the tube, Proteinase K was mixed as same as the previous tests, and then formamide (final 50%) was also mixed to the solution. Using formamide is a well-known method for in situ hybridization of DNA, which basically reduces melting temperature of double strand in a linear fashion by approximately 0.65° C. for each percent of formamide.

Triplicate tests showed no DASH pattern was observed using the samples after the formamide treatment. Compared to the successful redistribution results shown in the previous section, the results suggest that hybridization is playing at least part of the role in this generation process, in addition to physical entanglements by long DNA polymers.

### Example 4

### SEM Images of the DASH Patterns and Spherical Structures in the Fibrous Networks

### Met

A diameter of spherical structures found in DASH patterns were measured (Supplementary Figure S12) using total 30 points picked from the sample SEM image. average of 0.26±0.10 μm were obtained.

Estimated Critical Molecular Weight of ssDNA for the DASH Formation

Average ssDNA length synthesized by the reaction can be roughly estimated by following the technical specifications provided by the manufacturer. According to the manufacturer, 1 Unit of RepliPHI Phi29 can process 25 pmol of dNTPs in 30 min. i.e. 50 pmol of dNTPs will be incorporated into ssDNA in 1 hour. Typical reaction contains 5.7 U/μL of the enzyme with final 5 nM of generation seed concentration. The average length of ssDNA after 1 hour of synthesis based on this parameter would be:

$${N}_{( 1 hr . )} = \frac{50 \times {10}^{- 12 } mol / U \cdot 5.7 U \text{/} μL}{5 \times 1 {0}^{- 15} mol} = 5.7 \times 1 {0}^{4} nt$$

101641 For instance, typical minimum generation time required for the device #9-1 (FIG. 30) with the generation seed concentration of 5 nM was approximately 2 hrs. Thus, in this case, the average length of N_{(2hr.)}=1.1×10⁵ nt, i.e. molecular weight of 3.3×10⁷ (molecular mass of over 10 million Da), was calculated as a rough estimation of the critical molecular weight for the DASH formation under this condition.

### Example 5

### Control Experiments for the Sensitivity Analysis of Vorticity and Flow Velocity

To roughly understand the relation between DASH generation and the flow (rate, velocity, vorticity) at the side of pillars, several additional measurements and comparisons were made using actual experiments and CFD simulations using the devices with the same pillar geometry.

### Experiments Using 3-Chamber Device

A three-chamber device (FIG. 38, #12-1) was used to determine the relationship between flow rate and DASH generation starting time. All three chambers share the same pillar dimensions (same as #3-1); the widths of the main chambers (narrow: 175 μm, medium: 385 μm, wide: 805 μm were the only difference. The widths were decided based on the maximum image capture size of the microscope. This design allowed simultaneous DASH generation test at three different flow rates with the same pillar design in one trial. Three flow rates (slow: 0.1155 μA/min., middle: 0.231 μL/min., fast: 0.462 μL/min.) were selected in both simulations and in actual experiments. The middle flow rate was set in order to have equivalent flow velocity of the standard experiments (with 1-inlet devices) at the middle chamber (e.g. device #3-1 (500 μm width) with 0.1 μL/min. flow rate (approximately between 0.2-0.5 mm/sec)). CFD simulation results successfully showed the difference of flow velocity and vorticity inside the device corresponding to the width of chamber and flow rates, as intended (FIGS. 10A-10C and FIGS. 11A-11C).

### Relation Between Vorticity/Velocity and DASH Generation Starting Time

The average vorticity at the side of pillars were compared to the starting time of DASH generation taken from actual experiments. Actual experiments were tested by setting three different flow rates mentioned above using #12-1 device with a 5 nM generation seed concentration; each generation process was measured by a fluorescence microscope (150 sec/frame). A signal-to-noise ratio (SNR) calculations of the generated DASH patterns based on 1-D Fourier transform were executed row-to-row, chamber-to-chamber, and frame-by-frame, then the sample of 6 consecutive rows (row no. 213 to 218) in each chamber were selected, and average value is used as sample data representing DASH generation process of each condition (FIG. 12). The SNR value of 2.0 was set as an arbitrary threshold to determine generation starting time points quantitatively; the time point (frame no.) that surpassed the threshold in each sample was recorded as the starting frame of DASH generation. Note that “characteristic” flow velocities (μm/s) inside the device were used as a legend in the graph; the values were simply calculated based on a cross-sectional size of the chambers (W×H (μm²); W=chamber width, H=17.4 μm and the flow rate (μL/min.). Each characteristic flow velocity under the respective condition was then converted to vorticity by CFD simulation. The plot between characteristic flow velocity and vorticity showed a clear correlation between two values.

Based on these data, a comparison between vorticity and DASH generation starting time was plotted. The graph roughly showed a correlation between vorticity and inverse of the generation starting time (1/frames), suggesting vorticity and the DASH generation starting time have inverse correlation (i.e. higher vorticity results with faster generation starting time).

### Example 6

### Vorticity Comparison Between Different Shapes of Pillars

### Simulation

CFD simulation of various device geometries were tested based on the protocol mentioned in Materials and Methods (Example 2). In particular, square pillar (FIG. 28, 43-1) and rhombic pillar (FIG. 39, #3-2) devices share the same overall geometry of the device (including the same periodicity between the pillars, numbers of the pillars, and the same width of the pillars), but with different shapes of pillars. As a result, the overall flow velocity became almost identical (FIGS. 13A-13B), but the magnitude of vorticity at the side of the pillars became drastically reduced with rhombic pillars compared to square pillars (FIGS. 14A-14B) due to its streamlined shape. This difference between two geometries can be used as a model case for the comparison that whether the difference in the degree and the size of high vorticity region at the side of pillars affected the DASH generation process.

### Generation Time Comparison Experiment

To investigate the effect due to the difference of vorticity, signal-to-noise ratio (SNR) of the DASH patterns generated from both square and rhombic pillar devices were experimentally measured by the time-lapse observation during the process. The values quantitatively represent the “strength” of the generated DASH patterns (1-D line patterns) observed at each frame (higher S/N represents the stronger pattern generation). The tests were executed with triplicates (Red: square pillars, Blue: rhombic pillars) with the same experimental conditions (generation seed cone. of 0.5 nM, flow rate of 0.1 μL/min.

The results (FIG. 15) showed that there is a clear tendency in the difference between two types of pillar shapes; the devices with square pillars generated DASH patterns faster than the rhombic pillar devices, even with the same other parameters including flow velocity and seed concentration. The result suggests that the difference of vorticity at the side of pillars affected the generation process (higher vorticity brings faster generation).

### Example 7

### Generation—Degeneration of the DASH Patterns

### FSA Representation

Sequential generation and degeneration behavior are described as a finite state automaton (FSA), a mathematical model commonly used in robotics, systems engineering, and computer science (FIG. 3B).
M: Q={Init, Growth, Decay}.
Σ={Generation initiated, Flow altered, Digestion completed}
F={Init}

Here, Q represents the set of the states (behaviors). Σ is the set of input stimuli (the releasers of each behavior), F is the final state. The model allows approximation of the overall behavior in a discrete manner using three different states, such as Init, Growth, and Decay. Init represents the initial state without any DASH generation; all three solutions flowing into the device were remained laminar. DASH pattern generation triggers the state transition to Growth state. During this state, the degeneration mix was still kept separated from the generation mix due to laminar flow, so the anabolic process occurs. Once the generated DASH pattern started to fill the gap between pillars, the flow is altered by this physical feedback, thus the state transition occurs and changes to Decay state. Due to the mixing of generation and degeneration solution, the catabolic process dominates inside the device thus the pattern degenerates. When digestion is completed, the state returns to the original Inn state and can repeat the loop if DNA synthesis time is kept constant.

### Detailed Description of the Experimental Results

Time-lapse video was recorded using the standard protocol. The average fluorescence intensity plot in the main text (FIG. 3C) was plotted by measuring the average intensity at row 837, between columns 120 to 190 (which crossovers with multiple segments of the DASH patterns). To further show that the generation/degeneration occurred in a synchronous fashion in multiple locations, for example, fluorescence intensities from three sample points were plotted. Single intensity peak around 200-250 min. was observed with all three sample points, which corresponds well with the average fluorescence intensity mentioned above. The points were selected from three different segments of the DASH patterns. In addition, average intensity of the rectangular area was also calculated (which contains two segments of the DASH patterns), to further show the behavior is not due to the anomalistic behavior of specific sample points, but because of the overall generation/degeneration behavior. Triplicate recordings from three different DASH devices successfully repeated the similar tendency. In addition, negative control test (degeneration mix without DNase I) showed no decay behavior. The results show that the DASH patterns were generated (until around 250 min), and then completely degenerated in a synchronous fashion due to DNase activity.

### CFD Simulation

Here we simulated the behavior of the flow inside the DASH device according to the development of the DASH patterns with controlled accumulation (Supplementary Figure S24); to simplify the setup, we traced the accumulated region from experiments, regarded as a solid region, and performed CFD simulations. Particle-trace model (Particle density: 1.34 g/cm³, Particle radius: 13.8 μm, Coefficient of restitution: 0.5) was applied. Particles from the center inlet (Inlet 1; generation mix) were colored with black; particles from sides were colored with red.

First, laminar flow creates two distinctive regions inside the channel (FIG. 16A), so that the effect of degeneration solution from sides were kept minimum during the generation process at the region with black panicles (generation mix), thus the DASH patterns were generated at the center region of the device in the actual experiments. Once the controlled accumulation occurs at the center of the device (FIG. 16B), this structural change start to alter the solution during the development, and causes the mixture of black and red particles (i.e. mixture of generation and degeneration solution) (FIG. 16C and FIG. 16D). As a result, degeneration mix exceeds generation in the mixed region and thus the DASH patterns were digested. The CFD simulation clearly showed that the switch of states from growth to decay is due to the spatio-temporal feedback triggered by controlled accumulation of the DASH patterns.

### Repeated Generation—Degeneration

Similar to the generation/degeneration experiments, repeated generation/degeneration of the DASH patterns at the static location was tested using the device #20-2. Note that here we controlled the synthesis time to constant 2 hr. reactions, in order to minimize irreversible accumulation inside the device and to allow repeatable redistribution process throughout 12 hr. observation. As same as the other experiments, this experiment is also performed without human manipulation/intervention; all generation/degeneration reactions were executed autonomously.

The tests were tried with final 0.1 nM of generation mix solution. The graph (FIG. 17) shows the overall behavior by taking an average at the row 822, between columns 135 to 165 (which crossovers with multiple segments of the DASH patterns). In addition, to show the synchronous behavior at different locations, three sample points were chosen from time-lapse video, and the intensity were plotted. Furthermore, to show the phenomenon is not due to the anomalistic behavior of specific sample points, an average from the rectangular area that contains DASH patterns (one fiber segment) was also plotted. All results showed overall consistency in two cycles of generation and degeneration, by showing two peaks during 12 hr. test. Similar results were also repeated by using different concentration of generation mix (0.5 nM).

### Example 8

### Emergent Locomotion Behavior Powered by DASH Analysis

The inventors quantitatively analyzed the locomotion of the body by two parameters, such as center of mass plot (CoM) and perimeter detection. CoM represents the overall migration of the mass in the image; Perimeter shows the continuous migration of the entity.

### CoM Plots

Center of Mass (CoM) of the DASH patterns during locomotion with two types of straight tracks (wide width: FIG. 40, #22-3 and narrow width: FIG. 41, #23-3) were plotted (FIG. 3D). An x-axis distance between the CoM and the left edge of the time-lapse images were plotted over the course of locomotion.

The initial decrease of the value (i.e. CoM moving towards the downstream side) is due to the initial development of the patterns at the left-most side (downstream) of the device. (The default location of CoM is at the center of the image, since no pattern generation was occurring inside the device; eventually CoM moved due to the initial pattern generation at the most downstream region.) Once the pattern migrating towards the upstream against the flow direction, CoM corresponded to the location of the pattern and accurately representing the movement. Once the pattern reached the right edge (most upstream region) of the device, CoM approximately “stopped” at that final location. The average velocity of locomotion was calculated as 1.2 m,/hr. (#22-3), and 2.3 mm/hr. (#23-3) from the initial and the final location of CoM.

### Perimeter Analysis

The body was defined as a DASH pattern which occupies the largest consecutive area in the channel. Based on this definition, perimeter analyses were calculated based on the following algorithm using MATLAB. First, the timelapse images from fluorescent microscope were loaded frame-by-frame using in-house software, then converted into binary (black and white) images using arbitrary threshold (0.015). “Holes” in the binary images were then filled to determine the region of the body. Finally, the largest occupied region in the image was selected frame by frame, then, the perimeter of the region was displayed as shown in the movie.

### Example 9

### Details of the DASH-Based Detection

DASH-based detection was designed using a combination of hybridization/ligation-based recognition, DASH pattern generation-based amplification, and DASH pattern recognition-based readouts (FIG. 18). Recognition process uses a template with complementary sequence of target DNA or RNA; to simplify the case, in this demonstration, negative control uses “incorrect” target sequence with 2 bp mismatch at the ligation site of the template. Only a combination of correct target and template ends up with the successful ligation (circularization) of templates, which allows enzymatic synthesis process for the next amplification step. Note that both amplification and readouts were simultaneously and autonomously performed by utilizing mesoscale pattern generation capabilities of DASH.

### Results

Triplicate samples from both positive and non-targets (from 5 pM to 500 pM) were observed using time-lapse recording for total 78 frames (150 sec./frame). In order to quantitatively represent the existence of the patterns inside the device, the generation results were analyzed and plotted using in-house FFT software (FIGS. 19A-19B and FIGS. 20A-20B). The comparison plot (FIG. 21) was generated by using the maximum value of Signal-to-noise ratio (S/N or SNR) of the generated. DASH patterns as one axis, and another axis by plotting a maximum value from the average fluorescence intensity inside the chamber (after subtraction of the background intensity). Here the SNR representation was used to quantitatively show the existence/non-existence of the patterns (i.e. to simulate naked-eye readouts in a quantitative fashion); when we set an arbitrary threshold of SNR=15, the results corresponded well with our qualitative observation results as shown in FIG. 4A. The plot also showed a clear comparison that our pattern-recognition based detection can improve the detection sensitivity more than 10 times (detectable with 500 pM and 50 pM) with the target concentration compared to the average intensity values (which were undetectable in both concentrations), and also can maintain its specificity compared to the negative control samples (target sequence with 2 bp mismatch).

### Example 10

### DASH-Avidin Hybrid Materials

The results show Avidin was successfully bound to DASH (FIG. 4B and FIGS. 22A-22B), but not with Streptavidin as a control experiment (FIGS. 23A-23B). Both avidin and streptavidin are well known for their ability to bind to the coenzyme biotin. However, there are distinct biochemical differences between these related proteins that explain the observed difference in affinity for DNA (including DASH patterns). First, avidin has a strongly basic isoelectric point of approximately 10, and as such has a net positive charge in the RepliPHI reaction buffer (pH 7.5). Thus, avidin is electrostatically attracted to highly negatively charged DNA. Streptavidin, on the other hand, has a pI of about 5 or 6 and therefore has a slightly negative net charge in RepliPHI reaction buffer. Beyond electrostatic attraction, avidin is glycosylated, while streptavidin is not contributing to an increase in nonspecific binding between avidin and a variety of substrates. In a previous study, the non-specific interaction between avidin and DNA was characterized in detail and shown to be of high affinity due both to the overall positive charge and unique structural motifs of the protein.

This avidin-based binding can be utilized as a standard functionalization method for DASH patterns via avidin-protein conjugate or biotin-conjugated molecules using avidin-biotin interaction. The inventors further demonstrated functionalization of DASH patterns by Quantum Dots and HRP based on this method.

### Example 11

### DASH-Quantum Dots Hybrid Materials

Based on the successful results of binding fluorescent-avidin conjugates to the DASH patterns, this technique was expanded as a versatile functionalization method of the DASH patterns. Quantum dot (Qdots) attachment to DASH patterns (DASH-Qdots) were tested by using “sandwich” binding method. First, avidin was attached to DASH patterns, and then Biotin-conjugated Quantum dots were attached to the structures by using avidin-biotin interaction.

### Positive and Control Experiments

Both positive and control samples were tested with triplicates to confirm the consistency of the results. Representative results from positive and control samples were shown (FIG. 4C and FIGS. 24A-24D), The results clearly showed that only the positive samples (with avidin and then Qdots) succeeded to bind Qdots to the DASH pattern, even after additional wash for 1 hr with 1× RepliPHI reaction buffer. On the other hand, no binding was observed with control samples (without avidin binding). The results show successful functionalization of Qdots, and also suggest the binding mechanism of Qdots is indeed based on avidin-biotin interaction. This result along with the results with HRP binding suggests that the functionalization of DASH by this “sandwich” method (using avidin, then followed by biotin-conjugated target molecules) can be expanded to wide range of molecules from organic (proteins) to inorganic (nanoparticles).

### Example 12

### DASH-AuNP Hybrid Materials

### Darkfield Microscope

Darkfield light microscope allows clear characterization of gold nanoparticles due to strong light scattering. Olympus BH-2 microscope (Japan) with darkfield setup was used for the observation of DASH-AuNP patterns (40 nm AuNP were used for the results shown below) (FIG. 4D and FIG. 25).

### SEM

uccessful AuNP-DNA attachment to DASH patterns were confirmed by SEM. Transferrable DASH device setup was used for the sample preparation. Gold nanoparticles were clearly attached along with the fibrous morphology of DASH structures; some shows “wire-like” ordered gold nanoparticles.

### Example 13

### Functionalization of the DASH patterns by avidin-HRP

Based on the successful binding experiments of fluorescent-avidin conjugates and biotin-conjugated molecules (via “sandwich” binding method) to the DASH patterns, the technique was further expanded by attaching other avidin conjugated functional molecules (an enzyme in this case), and tested whether the enzymes retained its biochemical activities on the DASH patterns. Here, avidin conjugated horseradish peroxidase (Avidin-HRP) was chosen as a model enzyme and attached to the DASH patterns; the enzyme activity was measured to show the successful functionalization of the DASH patterns.

The activity of HRP bound to the DASH pattern was first verified by using One-Step Ultra TMB-ELISA substrate solution from Thermo Fisher Scientific (Waltham, Mass.). The kit detects HRP activity by converting the TMB (3,3′,5,5′-Tetramethylbenzidine) substrate to a blue-colored (intermediate) complex (Amax=370 nm and 652 nm). The blue product of the TMB was found to directly stain DASH patterns possibly due to the electrostatic interaction of negatively charged DNA and the positively charged TMB product. The result can be also observed even by naked eyes.

Next, to further confirm that the HRP activity is localized at the DASH patterns, in situ HRP reaction was monitored by using QuantaRed Enhanced Chetnifluorescent HRP Substrate Kit from Thermo Fisher Scientific (Waltham, Mass.), QuantaRed Substrate uses ADHP (Acetyl-3,7-dihydroxyphenoxazine) chemifluorescence reaction, which converts from non-fluorescent compound to resorufin, a fluorescent compound with Ex./Em. of 570/585nm, by reacting with HRP. QuantaRed solution was infused to the device and continuously monitored by fluorescence microscope. During the process, it was observed that the product indeed started to develop corresponding to (and downstream of) the location of DASH patterns until the overall fluorescence became saturated throughout the device possibly due to high sensitivity of the reaction.

Cell-Free Protein Expression from the DASH Patterns

In addition to the quantitative measurement of expressed protein (FIG. 4E), direct observation of CFPE from DASH patterns was performed (FIG. 41E and FIGS. 26A-26B). The observation followed the standard protocol by using fluorescence microscope. The result shows successful sfGFP expression occurred only from the device with the DASH patterns.

What is claimed is:

1. A system for generating a material having an ordered structure and artificial metabolism, comprising
a device and a generation mix,
wherein the generation mix is a reagent comprising ingredients for forming a polymer, and
wherein the device comprises a main chamber designed to permit a directed flow of solution therethrough, the main chamber comprising obstacles to cause vorticity in a directed flow of a solution comprising the generation mix so as to initiate and promote assembly of polymers synthesized in the device to form said material.

2. The system of claim 1, wherein the main chamber comprises at least one inlet port and at least one outlet port to permit infusion of the solution comprising the generation mix into the main chamber through the at least one inlet port and flow from the at least one inlet port through the main chamber to the at least one outlet port.

3. The system of claim 1 or 2, further comprising a degeneration mix which comprises reagents for depolymerizing the polymer.

4. The system of claim 3, wherein the main chamber comprises at least two inlet ports for separately infusing a solution comprising a generation mix and a solution of a degeneration mix.

5. The system of claim 3, wherein the main chamber comprises three inlet ports, wherein the middle inlet port is for infusing a solution comprising a generation mix, and wherein the two outside inlet ports are for infusing a solution comprising a degeneration mix.

6. The system according to any one of claims 1-5, wherein the device comprises multiple main chambers.

7. The system according to any one of claims 1-6, wherein said material has a static pattern.

8. The system according to any one of claims 1-6, wherein said material as a mobile pattern.

9. The system of claim 8, wherein the pattern is a locomotive behavior, or a racing behavior between two locomotive bodies.

10. The system according to any one of claims 1-9, wherein the polymer is DNA.

11. The system according to any one of claims 1-9, wherein the polymer is RNA.

12. The system of claim 10, wherein the generation mix comprises dNTPs, a template nucleic acid, a primer, and a DNA polymerase.

13. The system of claim 12, wherein the primer and the template nucleic acid are annealed prior to being supplied to the main chamber.

14. The system of claim 12 or 13, wherein the template nucleic acid is a circular DNA.

15. The system of any one of claims 12-14, wherein the DNA polymerase is a Phi29 DNA polymerase.

16. The system according to any one of claims 10-15, wherein the degeneration mix comprises one or more nucleases.

17. The system according to any one of the preceding claims, wherein the generation mix comprises a reagent that produces a detectable signal.

18. The system according to claim 1 or 2, wherein the polymer is DNA, and the generation mix comprises (i) dNTPs, a template nucleic acid, and a DNA polymerase, (ii) dNTPs, a primer, and a DNA polymerase, or (iii) dNTPs, a template DNA, a primer, a DNA polymerase, and a ligase.

19. The system according to any one of the preceding claims, wherein the main chamber has at least a substantially planar shape.

20. The system according to any one of the preceding claims, wherein the main chamber has a dimension, along the direction of directed flow, in micron to millimeter scale.

21. A method for generating a material having an ordered structure and artificial metabolism, comprising
providing a device and a generation mix,
wherein the generation mix is a reagent comprising ingredients for forming a polymer, and wherein the device comprises a main chamber designed to permit a directed flow of solution therethrough, the main chamber comprising obstacles to cause vorticity in a directed flow of solution; and
supplying a solution comprising the generation mix into the main chamber and directing the flow of the solution through the main chamber, thereby allowing synthesis of polymers and assembly of the synthesized polymers to form said material.

22. The method of claim 21, wherein the main chamber comprises at least one inlet port and at least one outlet port, wherein the solution comprising the generation mix is infused into the main chamber through the at least one inlet port and is directed to flow from the at least one inlet port through the main chamber to the at least one outlet port.

23. A method for generating a material having an ordered structure and artificial metabolism, comprising
providing a device, a generation mix, and a degeneration mix,
wherein the generation mix is a reagent comprising ingredients for forming a polymer, and the degeneration mix comprises ingredients for depolymerizing the polymer, and
wherein the device comprises a main chamber designed to permit a directed flow of solution therethrough and to have obstacles which are spaced in a predetermined pattern and are of shapes and sizes to permit generation of vorticity in a directed flow of solution; and
supplying a solution comprising the generation mix and a solution comprising the degeneration mix to the main chamber of the device, and directing the flows of the solutions through the main chamber to allow the process of polymer synthesis and assembly and the process of polymer degeneration occur autonomously and in combination, thereby forming said material.

24. The method of claim 23, wherein the main chamber comprises at least two inlet ports for separately infusing the solution comprising a generation mix and the solution comprising a degeneration mix.

25. The method of claim 24, wherein the main chamber comprises three inlet ports, wherein the middle inlet port is for infusing a solution comprising a generation mix, and wherein the two outside inlet ports are for infusing a solution comprising a degeneration mix.

26. The method of claim 24 or 25, wherein the solution comprising the generation mix and the solution comprising the degeneration mix are infused into the main chamber simultaneously, sequentially, or in a predetermined order.

27. The method according to any one of claims 21-26, further comprisingvisualizing the pattern of the material generated.

28. The method of claim 27, wherein the visualizing is achieved by naked eye, a camera, a fluorescent microscope, a light microscope, or an electron microscope.

29. The method according to any one of claims 21-28, wherein said material has a static pattern.

30. The method according to any one of claims 21-28, wherein said material has a mobile pattern.

31. The method of claim 30, wherein the pattern is a locomotive behavior, or a racing behavior between two locomotive bodies.

31. The method according to any one of claims 21-31, wherein the polymer is DNA.

32. The method according to any one of claims 21-31, wherein the polymer is RNA.

33. The method of claim 31, wherein the generation mix comprises dNTPs, a template nucleic acid, a primer, and a DNA polymerase.

34. The method of claim 33, wherein the primer and the template nucleic acid are annealed prior to being supplied to the main chamber, and optionally wherein the template nucleic acid is a circular DNA.

35. The method of claim 33 or 34, wherein the DNA polymerase is a Phi29 DNA polymerase.

36. The method according to any one of claims 31-35, wherein the degeneration mix comprises one or more nucleases.

37. The method according to any one of claims 21-36, wherein the generation mix comprises a reagent that produces a detectable signal.

38. The method according to any one of claims 21-37, wherein the main chamber has a planar shape.

39. The method according to any one of claims 21-38, wherein the main chamber has a dimension in micron to millimeter scale,

40. A material made according to a method of any one of claims 21-39.

41. A method for detecting a nucleic acid of a pathogen, comprising:
providing a device, a generation mix, and a sample,
wherein the generation mix is a reagent comprising (i) dNTPs, a template nucleic acid, and a DNA polymerase, without a primer; (ii) dNTPs, a primer, and a DNA polymerase, without a template nucleic acid; or (iii) dNTPs, a template DNA, a primer, and a ligase, wherein the template DNA is circularized in the presence of said nucleic acid of a pathogen and said ligase,
wherein the device comprises a main chamber designed to permit a directed flow of solution therethrough, the main chamber comprising obstacles to cause vorticity in a directed flow of solution; and
supplying into the main chamber (i) a solution comprising the generation mix and the sample or (ii) a solution comprising the generation mix wherein the template DNA has been treated with the sample and the ligase to permit circularization of the template DNA if said nucleic acid of a pathogen is present in the sample; and
directing the flow of the solution through the main chamber, thereby allowing synthesis of polymers and assembly of the synthesized polymers to form a DASH material having an ordered structure and artificial metabolis when the nucleic acid of the pathogen is present in the sample.

42. The method of claim 41, wherein the main chamber comprises at least one inlet port and at least one outlet port, wherein the solution comprising the generation mix is infused into the main chamber through the at least one inlet port and is directed to flow from the at least one inlet port through the main chamber to the at least one outlet port.

43. The method of claim 41 or 42, wherein the main chamber has a dimension in micron to millimeter scale, and has a planar shape.

44. The method according to any one of claims 41-43, wherein the nucleic acid of the pathogen is DNA.

45. The method according to any one of claims 41-43, wherein the nucleic acid of the pathogen is RNA.

46. The method according to any one of claims 41-45, wherein the pathogen is a bacterium, a fungus or a virus.

47. The method according to any one of claims 41-46, wherein the DNA polymerase is a Phi29 DNA polymerase.

48. The method according to any one of claims 41-47, wherein the generation mix comprises a reagent that produces a detectable signal.

49. The method of claim 48, wherein the reagent is a fluorescent compound that binds to DNA.

50. A method of making a hybrid material, comprising:
generating a material having an ordered structure and artificial metabolism according to a method of any one of claims 21-39, wherein the polymer is DNA, and
infusing into the main chamber a solution comprising a reagent that binds to said material which is formed from assembled DNA, thereby forming a hybrid material wherein the material having an order structure and artificial metabolism formed from assembled DNA is bound with said reagent.

51. The method of claim 50, wherein the reagent comprises Avidin.

52. The method of claim 51, further infusing into the main chamber a solution comprising biotin conjugated with an enzyme (such as HRP) or with quantum dots.

53. The method of claim 50, wherein the reagent comprises gold nanoparticles.

54. A method of cell free protein expression comprising:
generating a material having an ordered structure and artificial metabolism according to a method of any one of claims 21-39, wherein the polymer is DNA, and
infusing a cell-free protein expression solution to the main chamber to allow production of a protein encoded by the DNA in the material.

55. A method for designing obstacles for a main chamber of a device for generating a material having an ordered structure and artificial metabolism, comprising:
defining a main chamber for generating the material having an ordered structure;
defining a pattern of the material to be generated therein; and
determining the sizes, shapes and positions of a plurality of obstacles in the main chamber of the device necessary to direct flow of a solution along shortest route within the main chamber and between adjacent obstacles.


SEQUENCE LISTING (12 sequences)

SEQ ID NO: 1 - DNA, 21 bp, Artificial Sequence
  oligonucleotide
gaccaccttcgcgtccaaagc

SEQ ID NO: 2 - DNA, 55 bp, Artificial Sequence
  oligonucleotide
cgaaggtggtctttttttttatatagaattctatatattttttttgctttggacg

SEQ ID NO: 3 - DNA, 21 bp, Artificial Sequence
  oligonucleotide
caaccaaacaccccaaccacc

SEQ ID NO: 4 - DNA, 55 bp, Artificial Sequence
  oligonucleotide,
gtgtttggttgttttttttttttttttttttttttttttttttttggtggttggg

SEQ ID NO: 5 - DNA, 33 bp, Artificial Sequence
  oligonucleotide
ctgagtgtgacctaggccggcatcattggatgc

SEQ ID NO: 6 - DNA, 33 bp, Artificial Sequence
  oligonucleotide
ctgagtgtgacctaggaaggcatcattggatgc

SEQ ID NO: 7 - DNA, 70 bp, Artificial Sequence
  oligonucleotide
gcctaggtcacactcagttttttttcggtgcgagtttacgctctacttttttttgcatccaatgatgccg

SEQ ID NO: 8 - DNA, 21 bp, Artificial Sequence
  oligonucleotide
gtagagcgtaaactcgcaccg

SEQ ID NO: 9 - DNA, 35 bp, Artificial Sequence
  oligonucleotide
ttttttttttttttttttttttttttttgctttgg

SEQ ID NO: 10 - DNA, 20 bp, Artificial Sequence
  oligonucleotide
caaaaaacccctcaagaccc

SEQ ID NO: 11 - DNA, 20 bp, Artificial Sequence
  oligonucleotide
taatacgactcactataggg

SEQ ID NO: 12 - DNA, 2569 bp, Artificial Sequence
  GFP
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggagaccacaacggtttccctctagaaataattttgtttaactttaagaaggagatatacatatgagcaaaggtgaagaactgtttaccggcgttgtgccgattctggtggaactggatggcgatgtgaacggtcacaaattcagcgtgcgtggtgaaggtgaaggcgatgccacgattggcaaactgacgctgaaatttatctgcaccaccggcaaactgccggtgccgtggccgacgctggtgaccaccctgacctatggcgttcagtgttttagtcgctatccggatcacatgaaacgtcacgatttctttaaatctgcaatgccggaaggctatgtgcaggaacgtacgattagctttaaagatgatggcaaatataaaacgcgcgccgttgtgaaatttgaaggcgataccctggtgaaccgcattgaactgaaaggcacggattttaaagaagatggcaatatcctgggccataaactggaatacaactttaatagccataatgtttatattacggcggataaacagaaaaatggcatcaaagcgaattttaccgttcgccataacgttgaagatggcagtgtgcagctggcagatcattatcagcagaataccccgattggtgatggtccggtgctgctgccggataatcattatctgagcacgcagaccgttctgtctaaagatccgaacgaaaaaggcacgcgggaccacatggttctgcacgaatatgtgaatgcggcaggtattacgctaggtgcggccgcagaacaaaaactcatctcagaagaggatctgaatggggccgcactcgagggtggcgatcagaacgcgaccggcggtcatcaccatcatcaccattaagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcggcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg